(19) United States
(12) Patent Application Publication
(10) Pub. No.: US 2017/0080166 A1
(43) Pub. Date: Mar. 23, 2017

(54) Device and System for Insertion of Penetrating Member

(71) Applicants: Kenneth Wayne Rennicks, Pearland, TX (US); James Patrick Herlihy, Houston, TX (US); Actuated Medical, lnc., Bellefonte, PA (US); Baylor College of Medicine, Houston, TX (US); Texas Heart lnstitute, Houston, TX (US)

(72) Inventors: Roger B. Bagwell, Bellefonte, PA (US); Ryan S. Clement, State College, PA (US); Andrew J. Meehan, Warriors Mark, PA (US); Maureen L. Mulvihill, Bellefonte, PA (US); Casey A. Scruggs, Middleburg, PA (US); Kevin A. Snook, State College, PA (US); William E. Cohn, Bellaire, TX (US); James Patrick Herlihy, Houston, TX (US); Kenneth Wayne Rennicks, Pearland, TX (US)

(73) Assignees: Actuated Medical, lnc., Bellefonte, PA (US); Baylor College of Medicine, Houston, TX (US); Texas Heart institute, Houston, TX (US)

(21) Appl. No.: 15/267,801

(22) Filed: Sep. 16, 2016

Related U.S. Application Data
(60) Provisional application No. 62/220,567, filed on Sep. 18, 2015.

(51) Int. Cl. A61M 5/42; A61M 25/06; A61B 8/08; A61B 17/34; A61M 5/32; A61M 5/46

(52) U.S. Cl. CPC A61M 5/427; A61M 5/3287; A61M 5/46; A61B 8/0833; A61B 17/3403; A61M 25/065; A61B 17/3423; A61B 2017/3413; A61M 2205/505

(57) ABSTRACT

A system, device and method for insertion of a penetrating member into tissue is disclosed, which may be handheld and automated. A detector obtains data regarding subdermal locations of tissue structures, including cavities such as blood vessels. A processor calculates the distance between a preselected target point below the tissue surface, such as within a blood vessel, and the tissue surface, and adjustment data for vertical, angular and extension adjustment of the penetrating member. Vertical, angular and extension actuators carry out the adjustments in real-time as calculated and directed by the processor. Changes in the location of the target point result in automatic recalculation and adjustment by the processor and various actuators. A vibrational actuator induces vibration to the penetrating member during insertion, overcome tissue deformation and vein rolling. A guidewire may be inserted through or by the device, for dilator and catheter insertion once the penetrating member is removed.

### CROSS-REFERENCE TO RELATED APPLICATIONS

The present application claims the benefit of co-pending U.S. Provisional Application Ser. No. 62/220,567, filed on Sep. 18, 2015, the contents of which are incorporated herein by reference in its entirety.

### FIELD OF THE INVENTION

The present invention relates generally to devices for penetrating tissues within a body for the delivery or removal of bodily fluids, tissues, nutrients, medicines, therapies, and for obtaining percutaneous access to body compartments (e.g., vasculature, spinal cavity) for secondary placement of medical devices (e.g., guidewires, catheters).

### BACKGROUND

Central venous catheters (CVCs) allow access to the central circulation of medical patients. More than 5 million CVCs are placed each year in the United States. The CVC is a key platform from which to launch a multitude of critical medical interventions for acutely ill patients, and patients requiring major surgeries or procedures. There are over 15 million CVC days per year alone in Intensive Care Units (ICUs) of US hospitals, and 48% of ICU patients have a CVC inserted at some point during their ICU stay. A CVC is also necessary for patients requiring urgent hemodialysis, such as in acute kidney failure, plasma exchange for various immune mediated diseases, multiple forms of chemotherapy for cancer patients, parenteral nutrition for patients whose gastrointestinal tract cannot be used for feeding, and many other medical interventions.

CVC placement has, since the 1950s, been performed using the eponymous technique developed by the Swedish Radiologist Sven-Ivar Seldinger. Using this technique a hollow bore needle, also referred to as an introducer needle, is advanced through a patient's skin and subcutaneous tissue and finally into a central vein, located millimeters to centimeters below the skin surface. The “central veins” are the internal jugular, subclavian, and femoral veins. Once the central vein is entered, a wire is manually place through the hollow bore needle and into the vein. The needle is then removed, and often a plastic co-axial tissue dilator is then run over the wire into the vein, then removed, also over the wire. This dilates the tissue around the wire, and allows smooth passage of a CVC, next placed over the wire and into the vein. Once the CVC is in place, the wire is removed, leaving the CVC in the vein.

Since the original description of the Seldinger technique, the standard guide for where to place the introducer needle through the skin has been the patient's surface anatomy. Veins are usually located, millimeters to centimeters below the skin, in specific relationship to certain surface landmarks like bones or muscles. However, CVC placement failure rates and the rates of serious complications such as arterial puncture, laceration, and pneumothorax or “collapsed lung” using surface anatomy have been reported to be as high as 35%, and 21% respectively, in well-respected studies. These failure rates are attributed to the fact that surface anatomy does not reliably predict the location of the deep central veins for every patient. In 1986, ultrasonography (US) was used to visualize veins below the skin surface and to use such images to more accurately guide the manual placement of CVCs. The use of this technique lowered the failure and complications rate for placement of CVCs to 5-10%. However, the ultrasound guided CVC placement technique requires substantial training and experience to perform reliably. As such, general and cardiovascular surgeons, anesthesiologists, critical care specialists, and interventional radiologists are typically required to place these catheters. Unfortunately, these specialists are often not available for placement of a CVC in the urgent or emergent time frame in which they are frequently required.

Even well trained, experienced providers can fail at the same rates to place a CVC due to factors that are not possible to account for, or are beyond their control, given the current state of insertion technique. Two premier factors are tissue deformity and venous wall deformation. When the introducer needle is pushed through the skin and subcutaneous tissues, the force can cause the central vein target to move from its original position, causing what is referred to as a “needle pass miss.” When a needle comes to the venous wall, it can also push the vein into a different position, called “rolling,” again causing needle pass miss. Needle pass misses can result in the needle hitting vital structures in the vicinity of the central vein such as arteries, lungs, or nerves and can cause serious complications. The vein wall can also be compressed by the force of the needle, causing the vein to collapse, making it nearly impossible to enter the vessel lumen and usually promoting passage of the needle through the back wall of the vessel, an event referred to as “vein blowing.” Vein blowing usually results in bleeding into the peri-venous tissue. Not only is bleeding a notable complication of and by itself, but it disrupts local anatomy usually precluding subsequent successful CVC placement.

Therefore, there has been interest in various alternative systems of CVC placement, including automated systems that any clinician or medical personnel could operate. Such a system could allow more widely available, reliable, and faster placement of a CVC, with lessened chance of complications. To this point, however, most investigation has focused on steerable needles to solve the fundamental challenges of tissue and vessel deformity. However, there has not been a satisfactory automated CVC placement system developed.

### SUMMARY OF THE INVENTION

An insertion device, system and method is disclosed combining actuated positional guidance for targeted placement with vibration of a penetrating member, such as a needle, for penetrating the skin, subcutaneous tissues and venous wall that mitigates the tissue and vessel wall deformity problems that plague needle insertion. The device and system includes a series of mechanical actuators that direct the path of the penetrating member, or needle, in accordance with a processor that calculates and directs the positioning and path of the needle placement. The various actuators may be automated for action as directed by the processor. Although described as being used for automated insertion of a penetrating member, such as a needle, the same device and system may be used to insert additional medical devices, including guidewires and catheters, within any body cavity, vessel, or compartment.

The insertion device employs the use of a specific vibrating penetrating member. Prior research has demonstrated that vibrating needles during insertion leads to reductions in both puncture and friction forces. This phenomenon is utilized in nature by mosquitos when they vibrate their proboscis to penetrate the skin of their host. The increased needle velocity from oscillation results in decreased tissue deformation, energy absorption, penetration force, and tissue damage. These effects are partly due to the viscoelastic properties of the biological tissue and can be understood through a modified non-linear Kelvin model that captures the force-deformation response of soft tissue. Since internal tissue deformation for viscoelastic bodies is dependent on velocity, increasing the needle insertion speed results in less tissue deformation. The reduced tissue deformation prior to crack extension increases the rate at which energy is released from the crack, and ultimately reduces the force of rupture. The reduction in force and tissue deformation from the increased rate of needle insertion is especially significant in tissues with high water content such as soft tissue. In addition to reducing the forces associated with cutting into tissue, research has also shown that needle oscillation during insertion reduces the frictional forces between the needle and surrounding tissues.

Therefore, adding oscillatory motion, also referred to herein as vibration and/or reciprocating motion, to the needle during insertion can overcome three challenges in advancing the needle tip to the desired location, as compared to the use of a static needle. First, tissue deformation between the skin and the target vein is minimized by the vibration. This tissue deformation and the “pop through” that occurs as the needle tip traverses different tissue layers can cause the target to move relative to the planned path of the needle. Second, the vibrating needle mitigates the rolling of the target vein. Third, the vibrating needle provides additional contrast in an ultrasound image for the user to observe the advancing needle and final placement location. Imaging modes that are particularly sensitive to velocity changes, such as ultrasound with color Doppler overlay, are especially sensitive in detecting vibrated needles.

The system also provides a way to change a target point before deploying the penetrating member. When the target point is changed, the processor recalculates and updates the positional information for the penetrating member, and provides updated adjustment data for the various actuators to perform, so as to align the penetrating member to the new target point. Imaging may be used with the insertion device, so that images of the subdermal area may be visualized and seen by a user. The target point may be selected and updated on the display by a user, for interactive control.

The insertion device may also be handheld for ease of use by a practitioner or user.

The insertion device and system, together with their particular features and advantages, will become more apparent from the following detailed description and with reference to the appended drawings.

### DESCRIPTION OF THE DRAWINGS

FIG. 1 is a perspective view of one embodiment of the insertion device of the present invention.

FIG. 2 is a side view of the insertion device of FIG. 1 and schematic diagram of placement for use.

FIG. 3 is a schematic diagram of the system for insertion of a penetrating member.

FIG. 4A is a side view of the insertion device of FIG. 2 showing adjustment of the handle.

FIG. 4B is a top plan view of the insertion device of FIG. 2 showing adjustment of the side arm for positioning.

FIG. 5A is a schematic diagram of the insertion device showing dimensions used for calculations by the processor.

FIG. 5B is a schematic diagram showing the target zone used for calculations by the processor.

FIG. 5C is an exemplary ultrasound display used in visually adjusting the insertion device.

FIG. 6 is side view of the insertion device of FIG. 1 showing schematic representations of the various adjustments directed by the processor for automated insertion.

FIG. 7 shows perspective view of the insertion device of FIG. 6 in partial cut-away to show the various actuators.

FIGS. 8A and 8B are side views showing the adjustment in the vertical direction by a vertical actuator.

FIG. 9 is a partial cut-away showing one embodiment of the vertical actuator for vertical adjustment.

FIG. 10 is a side view showing the angular adjustment by the angular actuator.

FIG. 11 is a partial cut-away showing one embodiment of the angular actuator for angular adjustment.

FIGS. 12A and 12B are exploded views of the portion of the insertion device having an angular actuator, showing a keyed relationship of the angular actuator from opposite directions.

FIG. 13 is a side view showing the adjustment by linear extension.

FIG. 14 is a partial cut-away showing one embodiment of the extension actuator for extension.

FIG. 15A is a top view in partial cross-section showing the extension actuator and connected extension shaft in a retracted position.

FIG. 15B is a top view in partial cross-section showing the extension actuator and connected extension shaft of FIG. 15A in an extended position.

FIG. 16A is a partial cut-away showing one embodiment of the vibrational actuator for vibrational motion.

FIG. 16B is a cross-section of one embodiment of the vibrational actuator for vibrational motion.

FIG. 17 is a perspective view of another embodiment of the insertion device including a guidewire for insertion.

FIG. 18A is a perspective view in partial cut-away of the embodiment of FIG. 17 showing a guidewire actuator for guidewire placement.

FIG. 18B is a perspective view in partial cut-away of the embodiment of FIG. 17 showing guidewire positioning through the insertion device.

FIG. 19A shows a perspective view of one embodiment of the embodiment of FIG. 17 showing the guidewire housing attached.

FIG. 19B shows an exploded view of the embodiment of FIG. 19A showing the guidewire housing detached.

FIG. 20A is a perspective view of another embodiment of the insertion device in which reciprocating motion and the vibrational actuator is inline with the penetrating member.

FIG. 20B shows a partial cross-section of the embodiment of FIG. 20A showing a guidewire passing through the vibrational actuator.

FIG. 20C shows a close-up of the cross-section of FIG. 20B.

FIG. 21A shows a perspective view of one embodiment of an inline housing having a sideport.

FIG. 21B shows a cross-sectional view of the embodiment of FIG. 21A.

FIG. 22 shows another embodiment of the neck having a plurality of sideports.

Like reference numerals refer to like parts throughout the several views of the drawings.

### DETAILED DESCRIPTION

As shown in the accompanying drawings, the present invention is directed to an insertion device, system and method that permits subcutaneous access to body cavities, such as blood vessels, for needle insertion and potential placement of guidewires, dilators, catheters such as CVCs, and the like. The device and system includes a plurality of actuators that may be automated for adjusting the position and deploying a penetrating member into the tissue of a subject, such as the skin of a patient. A target point is preselected and used to calculate the position and adjustments to the penetrating member, and the series of actuators are adjusted to control the various components of the device to produce the proper alignment so as to reach the preselected target position upon deployment. The actuators may be adjusted automatically based on calculations made by a processor, and may further be adjusted as the target point location is changed. In at least one embodiment, an image-based modality is used to obtain data on the tissue or cavity to be targeted. The entire device is preferably handheld for ease of use.

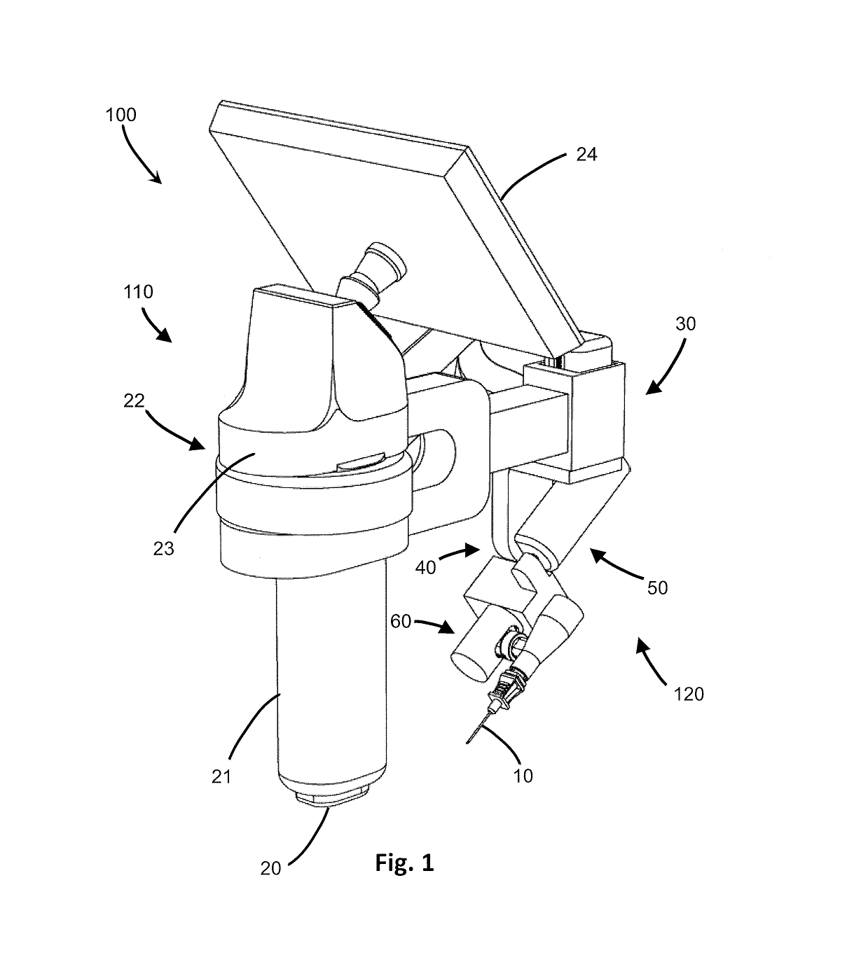

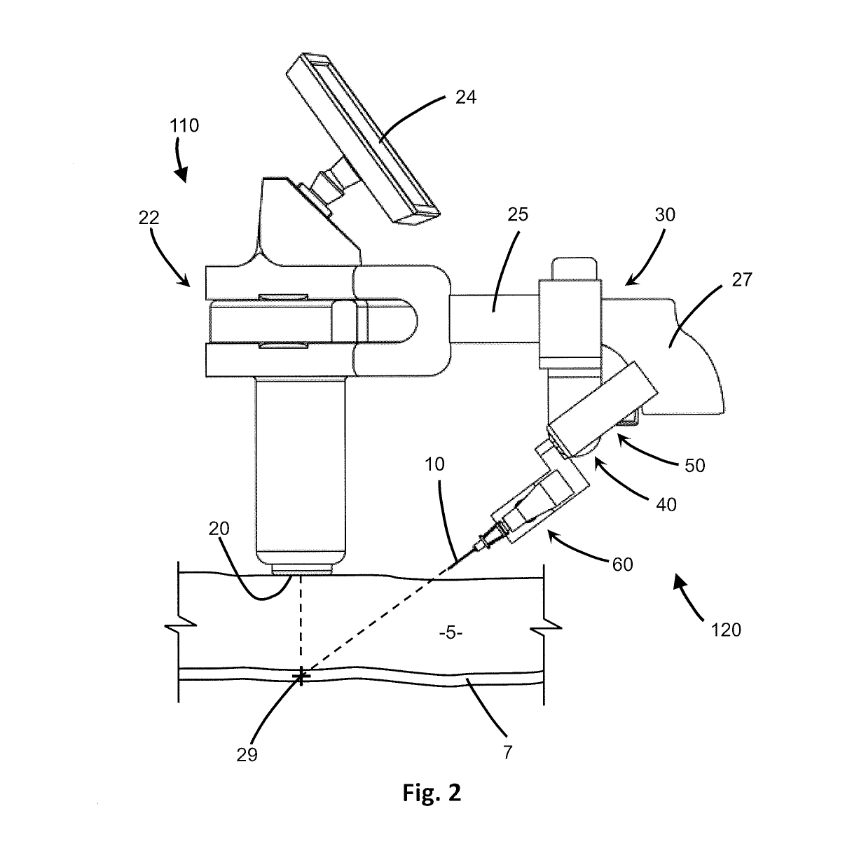

The insertion device 100, such as shown in the embodiments of FIGS. 1 and 2, includes a detector 20 to obtain data and information on the tissue of a subcutaneous area, a processor 22 to use this data to calculate various positioning and adjustment parameters for a penetrating member 10, such as a needle which may be an introducer needle, for insertion to a desired preselected target point 29 within the tissue based on the calculated parameters. The target point 29 may be any point located subcutaneously within a patient, such as in a blood vessel. Identifying the target vessel is a skill typical of many trained medical professionals in the healthcare industry. Guiding a needle to that target is the challenge, however, given the complications and risks to the patient from tissue deformation and vein rolling.

In at least one embodiment, the insertion device 100 allows the user to obtain information about a target vessel within tissue through an imaging modality, such as by ultrasound, and select a target point 29 on a display 24 showing a corresponding image of the vessel. The target point 29 can be adjusted on the display 24 by a user, such as on a touch screen, and a processor 22 automatically calculates the resulting height, trajectory, angle and distance the tip of a penetrating member needs to travel from its current location to reach the targeted location within the patient. Using these calculations, the processor 22 provides operative data or instructions to various actuators 32, 42, 52 of the positioner 120 to move the tip of the penetrating member 10 in various directions in an automated fashion to arrive at the desired position ready for deployment. Each actuator 32, 42, 52 may include sensors that send positional information to the processor 20 to be used in making the adjustment calculations. Once the desired position is achieved, the device 100 may be actuated to deploy the penetrating member 10 to advance the calculated distance. The processor 22 may also instruct the penetrating member 10 to automatically stop once it reaches the preselected target point 29 so that it does not go past the target point 29. The processor may also provide instructions to a vibrational actuator 62 to initiate and induce vibrating, such as reciprocating, motion to the penetrating member 10 during deployment to overcome the tissue deformation and vein rolling complications typically encountered in needle insertion.

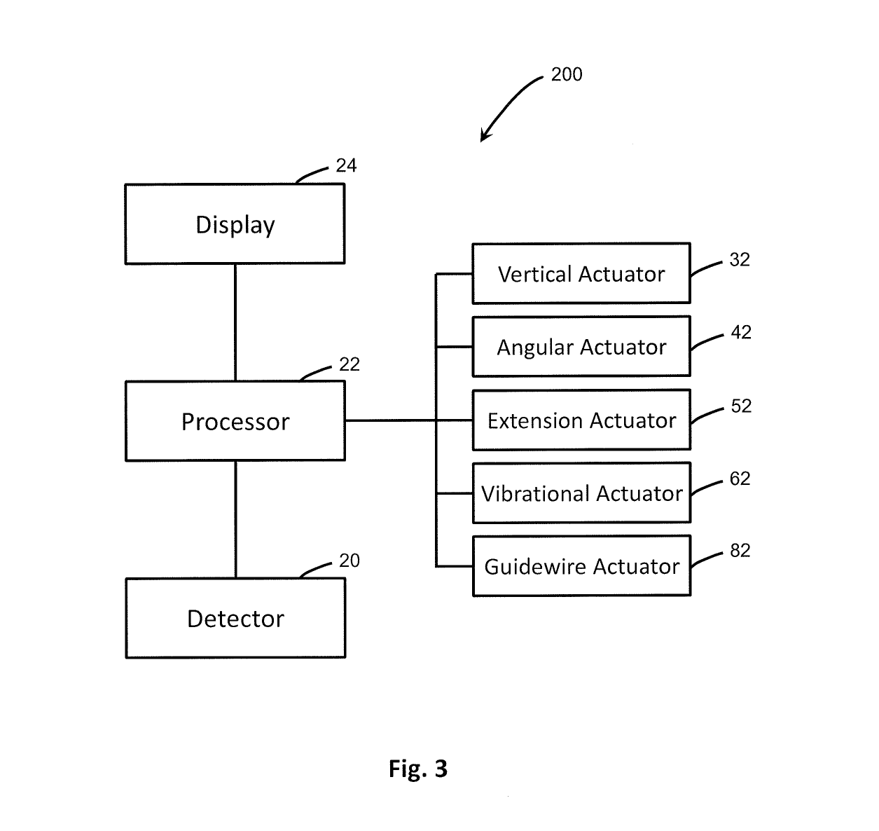

As seen in FIG. 3, the insertion device 100 also includes a system 200 in which information or data representative of the tissue below the surface, including cavities such as blood vessels, is obtained by a detector 20. In some embodiments, these data are images obtained by the detector 20, which may be an imaging detector. The data of the tissue beneath the surface are transmitted to a processor 22, which calculates the distance between a preselected target point 29 within the tissue or body cavity and the tissue surface. Computational software, logic circuits, and the like of the processor 22 uses this calculated distance to calculate adjustment data for vertical actuator 32, angular actuator 42, and extension actuator 52 and transmits this data to the corresponding actuator for movement of the penetrating member 10. The processor 22 also determines vibrational data for a vibrational actuator 62 based on the operative parameters of the actuator 62, and transmits this data to the vibrational actuator 62 for activation and inducing vibrational or reciprocating motion in the penetrating member 10 for deployment. Transmission of data to and activation of the various actuators 32, 42, 52, 62 may occur in any order or in a predetermined or defined order as set forth by the processor 22. The penetrating member 10 may be deployed automatically based on the extension adjustment data sent to the extension actuator 52. In some embodiments, a user decides when the appropriate positioning for the penetrating member 10 has been reached to align with the projected path to intersect the target point 29, and he/she may activate a deployment command, which is transmitted to the processor 22 and relayed on to the extension actuator 52, which extends the penetrating member 10 by a pre-calculated distance to the target point 29 below the skin based on the information from the images obtained.

In some embodiments, the detector 20 is an imaging detector, such as an ultrasound probe or other transceiver. The data obtained by the detector 20 may be presented on a display 24, which can be viewed by a user. A representation of a pre-selected target point 29′ may be overlaid on the image presented on the display 24, and may be moved around by a user. In at least one embodiment, the user may interact with the image or representations on the display 24, such as through an interactive touch screen or joystick, to move the representative target point 29′ around on the display 24. As the representative target point 29′ is moved on the display 24, the processor 22 calculates updated adjustment data for the vertical actuator 32, angular actuator 42, and extension actuator 52 based on the new representative target point 29′. This may be performed any number of times before a final target point is decided by a user, at which point the user may decide to deploy the penetrating member 10 for insertion and the corresponding instruction is sent to the extension actuator 52.

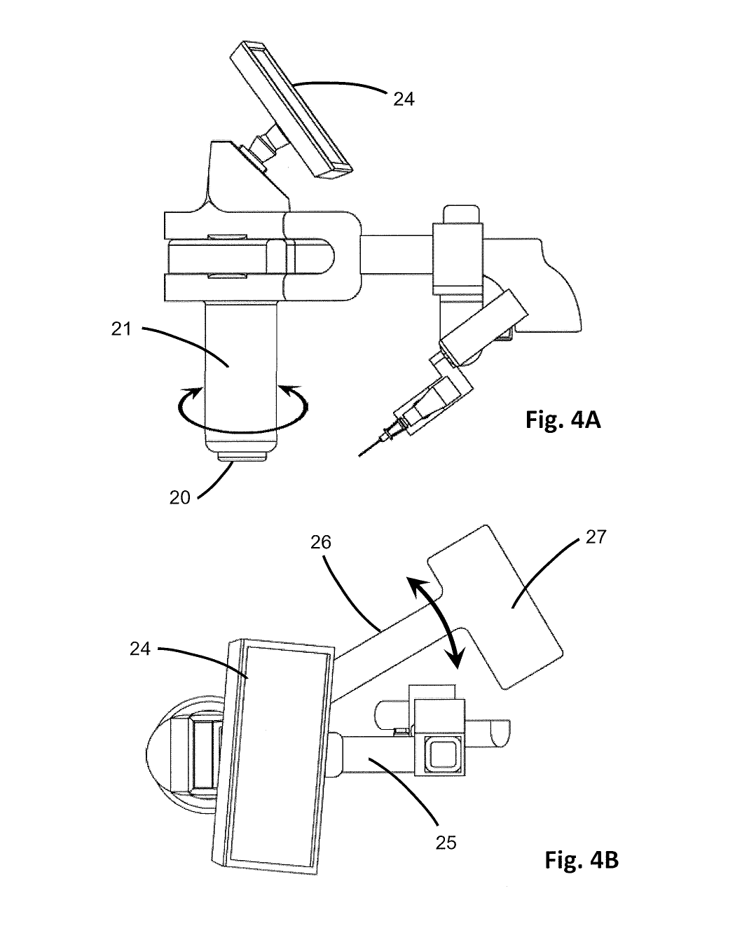

In use, the insertion device 100 is placed alongside or adjacent to the tissue, such as skin, of a patient in order to locate a target vessel, such as a vein. In at least one embodiment, as in FIGS. 1 and 2, the device 100 is handheld and includes a handle 21 which may be gripped by a user, such as a clinician or medical personnel. The handle 21 may be ergonomically shaped for increased efficiency and comfort in holding, particularly for a prolonged period of time if necessary. The handle 21 is preferably gripped by the non-dominant hand of a user, such as in the left hand of a right-handed person, to leave the dominant hand available for selecting a target location and deploying the device 100. Accordingly, the device 100 can be used equally by right-handed and left-handed individuals, and is not specific to grip direction. Indeed, in some embodiments the handle 21 may be rotatable about an axis, as shown in FIG. 4A, to accommodate different grip orientations or positions or to obtain different image views when imaging.

In at least one embodiment, the insertion device 100 also includes a support 27 which may be positioned in the elbow, shoulder, arm or chest of the user. The support 27 provides additional stability for a user when positioning and using the device 100. As depicted in FIG. 4B, the support 27 may be spaced apart from the handle 21, such as by a side arm 26 that corresponds to a user's arm, and may be adjustable in length to accommodate a user's reach. The side arm 26 may be movable in an arcuate path, as indicated by the directional arrow in FIG. 4B, to adjust the angle of the side arm and permit a user positioned next to a patient to comfortably use the insertion device 100 while properly aligning it as desired to target a vessel. The range of motion for the side arm 26 may be up to 360°, and therefore may permit any desired angle of approach. For example, a user may sit or stand adjacent to the patient and perpendicular to the desired target blood vessel, and yet the insertion device 100 may still be used to position the penetrating member 10 in alignment with the target blood vessel. The full range of motion of the side arm 26 may also permit switching from right-handed to left-handed use.

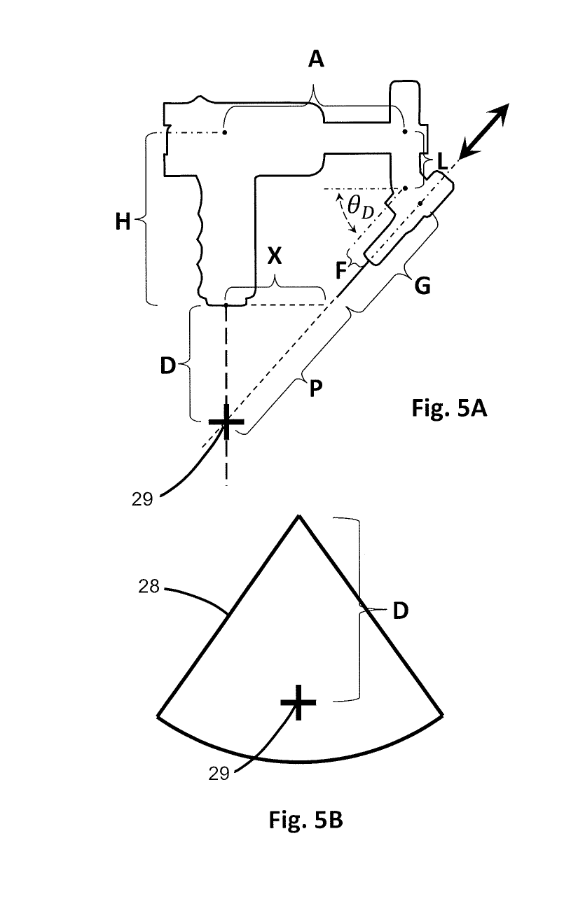

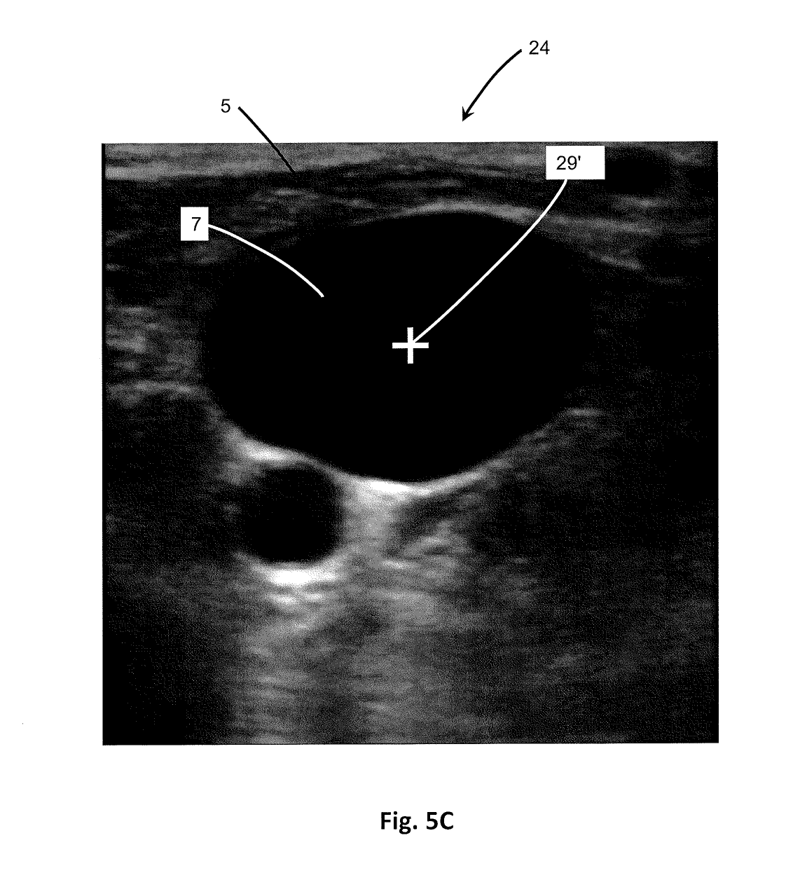

The insertion device 100 includes a detector 20 which is placed near, adjacent to, or even touching the area of the patient to be imaged, such as depicted in FIG. 2. In at least one embodiment, the detector 20 is located at a terminal end of the handle 21, such that the detector 20 may be positioned along the skin or other tissue 5 of a patient by moving the handle 21 over the patient. The detector 20 obtains information or data about the surrounding area, such as the subdermal area, and may including locational information of the tissue 5, cavities 7 and other structures therein. In at least one embodiment, the detector 20 is of an imaging modality to visualize a subcutaneous or percutaneous area of a patient, also referred to as a target zone 28 as shown in FIG. 5B, for targeting a particular blood vessel or body cavity 7. The target zone 28 imaged may be any shape, volume, or depth D as the particular imaging modality is capable of producing. The imaging modality may be any suitable form of imaging the subdermal area of a patient, such as but not limited to ultrasound, computerized tomography, and magnetic resonance imaging. In a preferred embodiment, as shown in FIG. 5C, ultrasound is useful for its ability to provide images that clearly distinguish between tissue 5 and body cavity 7, such as the interior of a blood vessel, below the surface of the skin. As used herein, “tissue” may refer to any tissue or organ of the body, and refers specifically to substantive material having mass. For instance, tissue may refer equally to skin, muscle, tendon, fat, bone, and organ walls. In contrast, “body cavity” as used herein may refer to the cavity, open interior, lumen or volume of space within a tissue or organ, such as blood vessels, veins, arteries, and the like.

Therefore, in at least one embodiment, the detector 20 is an ultrasound transducer that emits and receives ultrasound waves through the skin and tissue of a patient for visualization. Typical B-mode ultrasound imaging may be used in the detector 20, though Doppler ultrasound could also be used to distinguish blood flows of different directions. Linear or curvilinear ultrasound transducers are preferable, though sector phased arrays may be used in some embodiments. The ultrasound detector 20 may operate in the frequency range of 3-15 MHz, but more preferably in the range of 6-10 MHz to provide a good contrast between resolution and depth of penetration of the ultrasound, since depth of penetration is inversely related to frequency. Highly accurate measurement of the pixel size is important as it relates to distance, or phase velocity of sound in tissue, for accurate placement of the penetrating member 10. The ultrasound detector 20 may be operated in a long-axis image plane view, where vessels are viewed longitudinally, or a short-axis view, where the vessels are viewed in cross-section and appear as circular structures in resulting images, as in FIG. 5C. Imaging in the short-axis view is preferable in at least one embodiment to better visualize the body cavities 7, which appear as black spaces against the tissue 5, shown in white. The short-axis view permits the depth of the blood vessel to be seen for determining optimal placement of a target point 29 so as not to blow the vein or vessel. In either view scheme, the image plane produced by the detector 20 is at a known angle relative to the various actuators, discussed below, for proper positioning accuracy and co-registration of the ultrasound image and penetrating member 10 spatial coordinates.

The insertion device 100 further includes a processor 22 in electronic communication with the detector 20, and receives the data obtained by the detector 20 regarding the location of tissue 5 and cavities 7 therein. In some embodiments, these data are arranged as images of the subdermal area obtained by the detector 20, and are transmitted to the processor 22 and to a display 24, such as a screen that presents the images for visualization by a user, as depicted in FIGS. 1 and 2. FIG. 5C shows an example of an ultrasound image obtained by the detector 20 as presented on the display 24. The display 24 also shows a pictorial representation of the target point 29′, such as with crosshairs, a target sign, or other symbol in conjunction with the images from the detector 20. The representative target point 29′ image on the display 24 may be moved around, such as up and down on the display 24, by a user. As the representative target point 29′ is moved, the positioning of the penetrating member 10 is adjusted, as described below, which may occur automatically and in real time. The display 24 may show additional information, including but not limited to parameters of the detector 20 (such as the frequency used), screen resolution, magnification, measurements or position information from the various components of the positioner 120 (discussed in greater detail below), and buttons or areas to activate various components of the insertion device 100.

The display 24 may be a passive or interactive screen. In at least one embodiment, the display 24 is a touch screen that may operate through a resistive mechanism, capacitive mechanism, or other haptic feedback mechanism. For instance, the representative target point 29′ on the display 24 may be movable by touch on the touch screen, such as by sliding a finger, thumb or selection device along the display 24 in a continuous path, or by touching the display 24 screen in discrete locations to select new positions for the representative target point 29′. In some embodiments, the display 24 and processor 22 may be included in a single device, such as a smart phone, personal digital assistant (PDA) or tablet computer that may be removably connected to the insertion device 100 through a wireless protocol such as Bluetooth® or through a wired, multi-pin connector. In other embodiments, the display 24 and processor 22 are included in a single device, which may be integrated with the rest of the insertion device 100. In further embodiments, the processor 22 is an integrated component of the insertion device 100, and may be located within a housing 23 as in FIG. 1, and the display 24 may be separately removable from the remainder of the insertion device 100.

In other embodiments, the display 24 is a passive screen, such as a monitor, and the device 100 may include a joystick or directional button(s) (not shown) to enable the user to guide the imaging assembly 110 and target the vein. The joystick or directional button(s) may output a direction signal to the processor 22 based on the orientation and inclination of the joystick lever, or the particular directional button(s) pressed or selected. The output signal from the joystick or directional button(s) controls the position of a representative target point 29′, such as a crosshair, shown on the display 24 such that the target point 29 image overlays the target location. In some embodiments, the joystick or directional button(s) may be located at or near the display 24, such as along the edges of the frame of the monitor. In other embodiments, the joystick or directional button(s) may be placed on the handle 21 to enable one-handed operation of the device 100 for imaging.

The processor 22 is in electrical communication with, receives information from, the display 24 on the location and change of location of the desired target point 29 as indicated by a user from interacting with the representative target point 29′ on the display 24, such as by touch screen interaction. The processor 22 includes program(s), software, logic circuits, or other computational abilities to calculate how to adjust the penetrating member 10 from its existing position to a position that will bring it to the target point 29 as indicated by the user-indicated information provided from the display 24 interaction.

For example, FIG. 5A shows a schematic representation of the insertion device 100 depicting various dimensions used in the calculations by the processor 22. Some of these dimensions are fixed dimensions of the device 100. For instance, H is the height of the handle 21 from the detector 20 to a center of the primary arm 25. The distance A is the length of the primary arm 25 from the center of the handle 21 to the center of the positioner 120, such as the vertical actuator 32. In some embodiments, A is a fixed length, such as when the primary arm 25 is of a fixed length. The size of the mounting for the penetrating member 10, and the length of the penetrating tip 10, such as a needle, collectively referenced as G, is also known and fixed. The distance between the mounting for the penetrating member 10 and the angular adjustment 30, F, also remains fixed.

Other dimensions of the calculations will vary. For example, D is the distance between the detector 20, located at the surface of the tissue 5 or skin, to the target point 29 within the body cavity 7, such as the interior of a blood vessel beneath the skin. D will therefore vary by patient, as well as which blood vessel is used as the target, how much tissue lies between the target blood vessel and the skin or surface on which the detector 20 is placed, and even the position of the target blood vessel and how full or compressed the blood vessel is. In at least one embodiment, the height L of the positioner 120 may be varied. In some embodiments, the height L of FIG. 5A may be pre-set before use such that it is fixed when the insertion device 100 is in use. Using this information, the microprocessor may determine the angle of inclination, θ_{D}, and the distance from the tip of the penetrating member 10 to the target point 29, P, using the Pythagorean Theorem and trigonometry. For instance, once way the calculations may be performed are as follows:

$$P = \frac{A + F \cdot sin θ D}{cos θ D} - G$$

(H+D−L)·cos θD−A·sin θD=F

Alternatively, the angle θ_{D} could be pre-set by a user, and the height L and distance P would be calculated using similar mathematical relationships.

Looking at it another way, and still with reference to FIG. 5A, the depth D forms one side of a triangle, distance X is the distance between the center of the detector 20 to the tip of the penetrating member 10 and forms a right angle with D and another leg of the triangle. The distance for insertion of the penetrating member 10 is P, which is the hypotenuse of the triangle, and is calculated by solving for P in the following equation:

D²+X²=P²

The angle of insertion θ_{D} is therefore calculated as:

$$cos θ D = \frac{X}{P}$$

Accordingly, there are many ways to perform the calculations based on the known constant dimensions and the variables. The above provide just a few examples. In other embodiments, height L may be adjustable and automated during the use of the insertion device 100, such as when a shallow angle, or acute θ_{D}, is needed. This may be the case if the target blood vessel is itself very shallow or partially collapsed, or if it is located superficially below the surface of the skin. In such illustrative embodiments, to achieve an appropriate angle, the height L may be increased to position the penetrating member 10 to reach the target point 29. The amount of height L increase or decrease is calculated in real-time by the processor of the processor 22 as the angle θ_{D} is also calculated for adjustment based on the information input at the display 24 by the user. For instance, as the user slides a finger up along the display 24, the target point 29 indicator also moves up and the angle θ_{D} is made shallower or more acute. Conversely, as the user slides a finger down along the display 24, the target point 29 indicator also moves down and the angle θ_{D} increases or becomes deeper. Sliding a finger along a touchscreen display 24 is just one embodiment. In other embodiments, knobs or dials can be used to move the representative target point 29′ up or down on the screen, which would correspond to adjustments in the angle θ_{D} as determined by the processor 22.

The processor 22 is also in electrical communication with a positioner 120 that is spaced apart from the imaging assembly 110 of the insertion device 100, such as by a primary arm 25. The primary arm 25 may be of any suitable length sufficient to space the penetrating member 10 from the detector 20 so that the penetrating member 10 can approach, and reach, the desired target point 29. The primary arm 25 may be adjustable, such as manually or automated such as with an actuator, but in at least one embodiment it is stationary and of a fixed length.

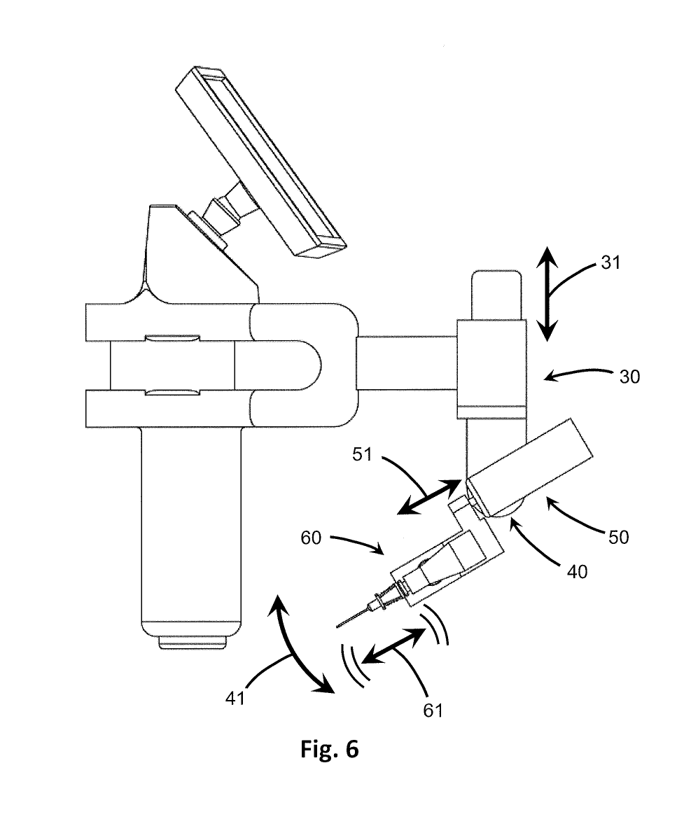

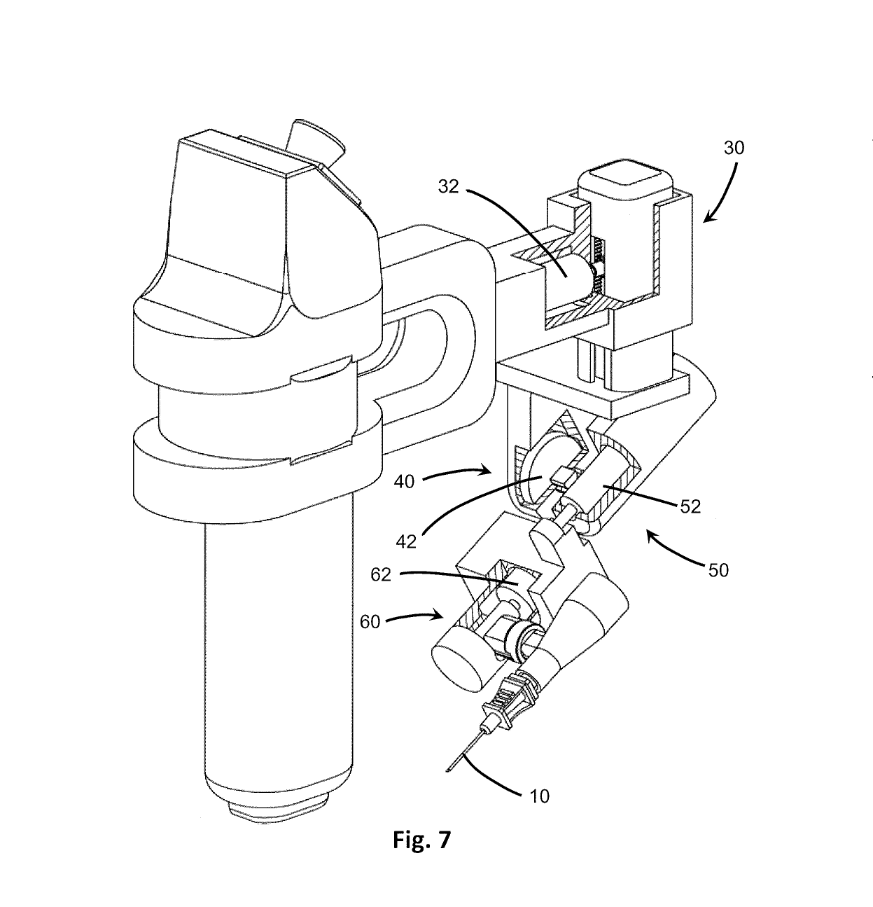

With reference to FIGS. 1, 2 and 6, the positioner 120 includes a vertical adjustment 30 that adjusts the penetrating member 10 in a vertical direction 31; an angular adjustment 40 that adjusts the angle of inclination of the penetrating member 10 along an angular direction 41; and an extension adjustment 50 that moves the penetrating member in a linear direction 51 toward or away from the target point 29 for insertion and removal. A vibrator 60 that provides reciprocating motion in a longitudinal direction 61 along the penetrating member 10 is also present in the insertion device 100, but need not be a component of the positioner 120. As seen in FIG. 7, each of the adjustment parameters is affected by actuators 32, 42, 52, 62 that receive signals from the processor 22 providing instruction on movement parameters and may automatically move according to those instructions to adjust the positioning of the penetrating member 10.

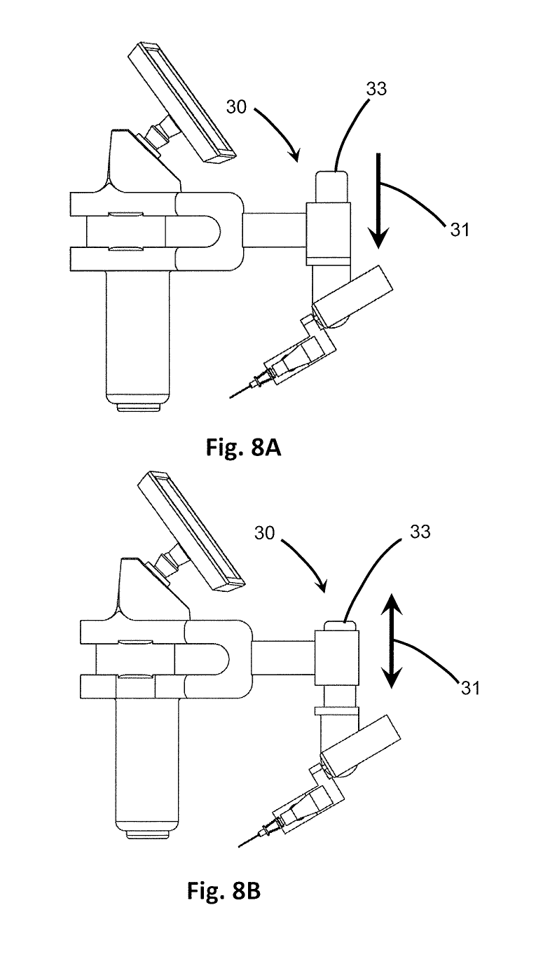

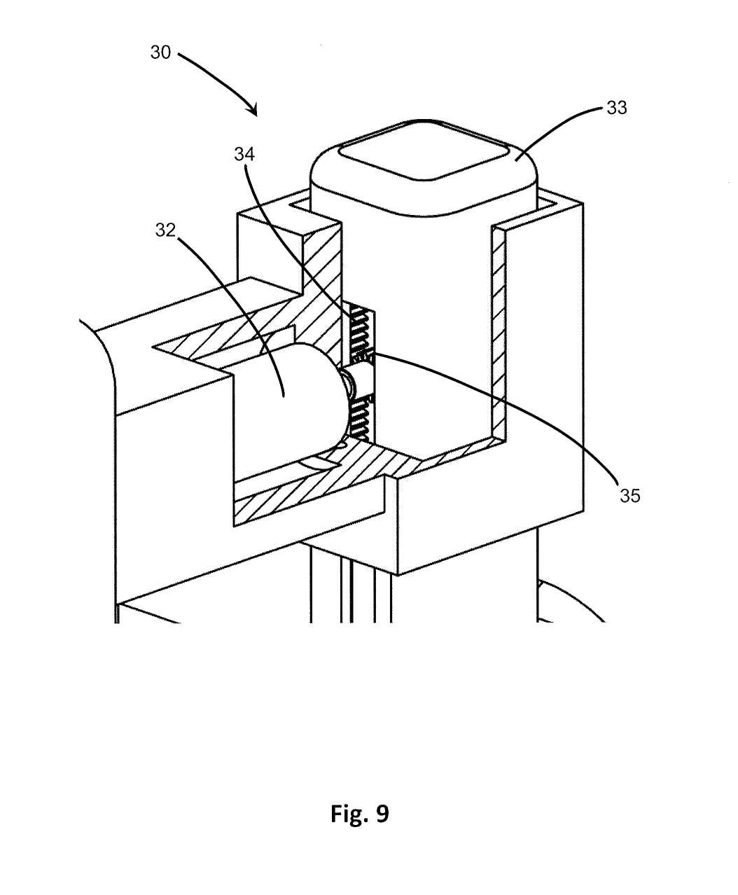

For instance, with reference to FIGS. 7-9, the vertical adjustment 30 provides a mechanism for raising or lowering the mounted penetrating member 10. Specifically, the vertical adjustment 30 includes a vertical actuator 32 which is in electrical communication with the processor 22 to receive vertical adjustment data for activation and movement. Upon receiving the signal or data from the processor 22, the vertical actuator 32 activates and moves according to the vertical adjustment data calculated by the processor 22 so as to adjust the penetrating member 10 in a vertical direction 31 with respect to the surface of the skin or other tissue being imaged for insertion. The vertical actuator 32 may be a motor that turns or acts on a shaft. For example, in at least one embodiment, as depicted in FIG. 9, the vertical actuator 32 is a rotational motor that turns a pin 35 which extends from the vertical actuator 32. The pin 35 engages a track 34, such as in an interlocking fashion between corresponding teeth or grooves on the pin 35 and track 34, such as in a rack and pinion system. As the pin 35 rotates in one direction, its extensions interdigitate with those of the track 34, and move the track 34 up or down in the vertical direction 31. When the vertical actuator 32 turns the pin 35 in the opposite direction, the track 34 is correspondingly moved in the opposite vertical direction. Accordingly, the vertical actuator 32 may be positioned perpendicular to the track 34. The track 34 may be located within a vertical housing 33. In other embodiments, the track 34 may be a slide bar, and the vertical actuator 32 may move a pin 35 between different locking positions along the slide bar to move the slide bar in the vertical direction. In still other embodiments, the vertical actuator 32 may be a linear motor disposed along the vertical direction 31, such that upon activation it causes a pin 35 or other elongate shaft to extend, thereby causing movement of the housing 33 in the vertical direction 31. As discussed above, in some embodiments, the vertical actuator 32 may be automated by the processor 22 and move in real-time as adjustments are made to the target point 29 at the display 24. In some embodiments, however, the vertical actuator 32 may not be activated, such as if adjustment in the vertical direction 31 is not needed or if the vertical height component is intended to be fixed.

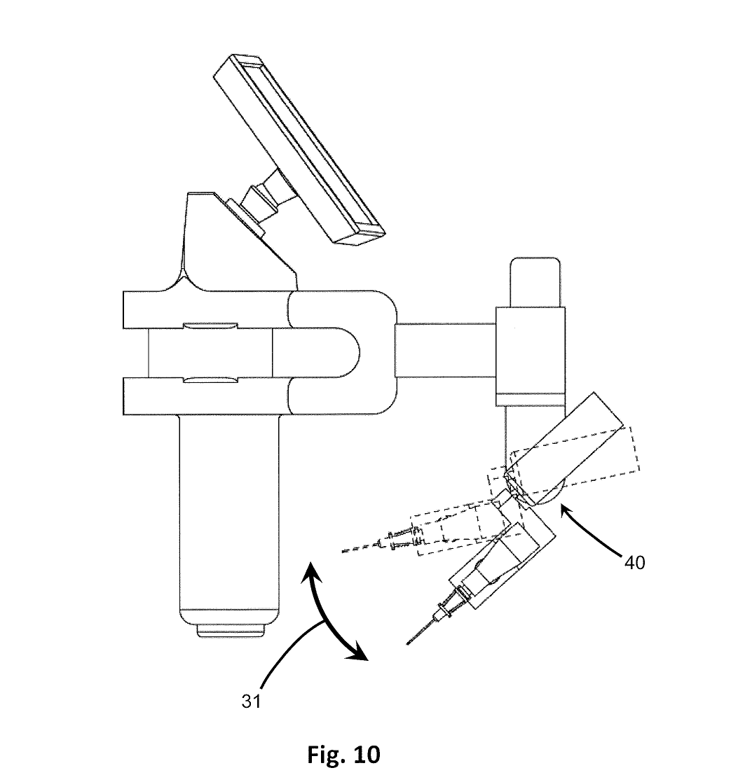

The positioner 120 also includes an angular adjustment 40, as depicted in FIGS. 7 and 10-12B. The angular adjustment 40 includes an angular actuator 42 in electrical communication with the processor 22. The angular actuator 42 receives signals, such as angular adjustment data, from the processor 22 providing instructions on activation for changing the angle of inclination of the penetrating member 10. The angle of inclination may be any angle between 0° and 180° with respect to the surface of the tissue. In at least one embodiment, the angle of inclination is an acute angle between 0° and 90°. The angle of inclination is adjusted in the angular direction 41 as seen in FIG. 10, according to the calculations performed by the processor 22. Accordingly, the angle for penetration can be made shallower or steeper as determined by a user. In imaging embodiments, when the user moves the representative target point 29′ up or down on the display 24, the corresponding signal is relayed from the processor 22, and the processor 22 updates the calculations to determine an updated or new angular adjustment data based on the new position of the representative target point 29′. This updated data is sent to the angular actuator 42, which activates to adjust the angle of the penetrating member 10 accordingly, which may be in real-time. This activation is automated by the processor 22. The angular actuator 42 may be a motor suitable for changing the angle of inclination. In a preferred embodiment, the angular actuator 42 is a rotational motor that rotates upon activation. In such embodiments, a shaft 43 extends from the angular actuator 42 into a receiver 45 or other structure not fixed and independently movable from the angular actuator. The shaft 43 and corresponding receiver 45 may be correspondingly shaped, such as being matingly fit or in a complimenting keyed arrangement, so that rotation of the shaft 43 imparted from the angular actuator 42 correspondingly turns the mating receiver 45.

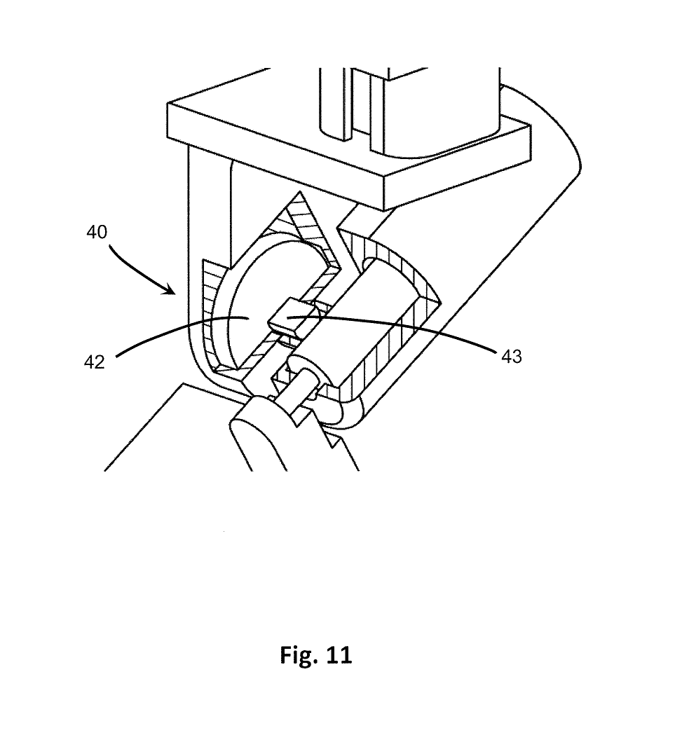

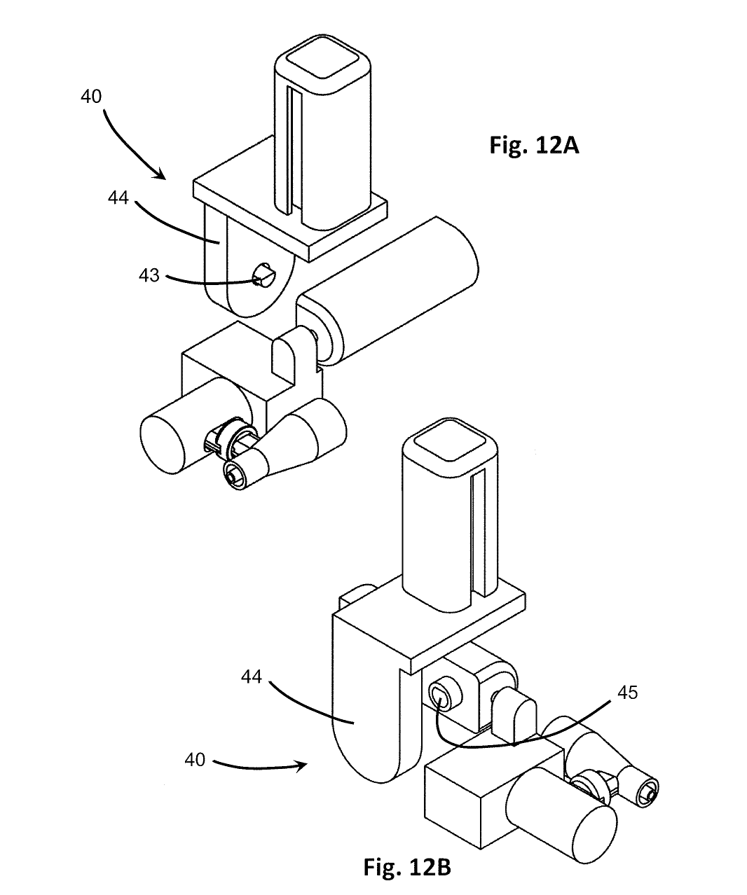

For example, in the embodiment of FIGS. 11, 12A and 12B, the shaft 43 has a keyed configuration such that it has an irregular shape, such as having a flat surface along one side of an otherwise cylindrical shape. The receiver 45 into which the shaft 43 extends is similarly keyed, having a flat surface along at least a portion of its perimeter. Accordingly, when the shaft 43 is rotated by the angular actuator 42, the specific shape engages the corresponding shape of the receiver 45 and transfers the rotational motion on to the receiver 45, thereby turning the receiver 45 as well. Since the receiver 45 is integral with a separate component of the positioner 120 from the angular actuator 42, the rotational motion conveyed to the receiver 45 through the correspondingly shaped interaction with the shaft 43 also turns the remaining portion of the positioner 120, as shown in FIG. 10. The angular actuator 42 may be surrounded by angular motor housing 44, which may include an aperture through which the shaft 43 extends, as seen in FIGS. 11 and 12A.

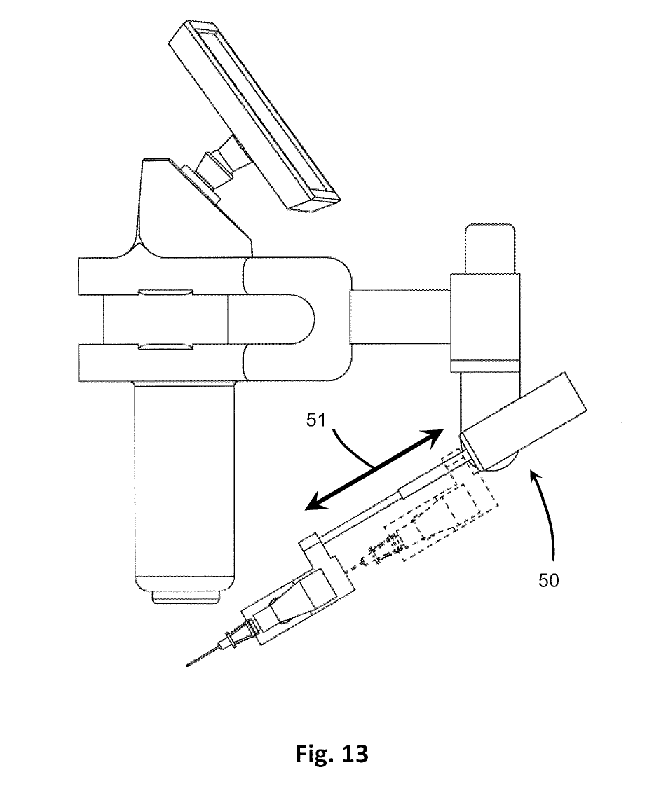

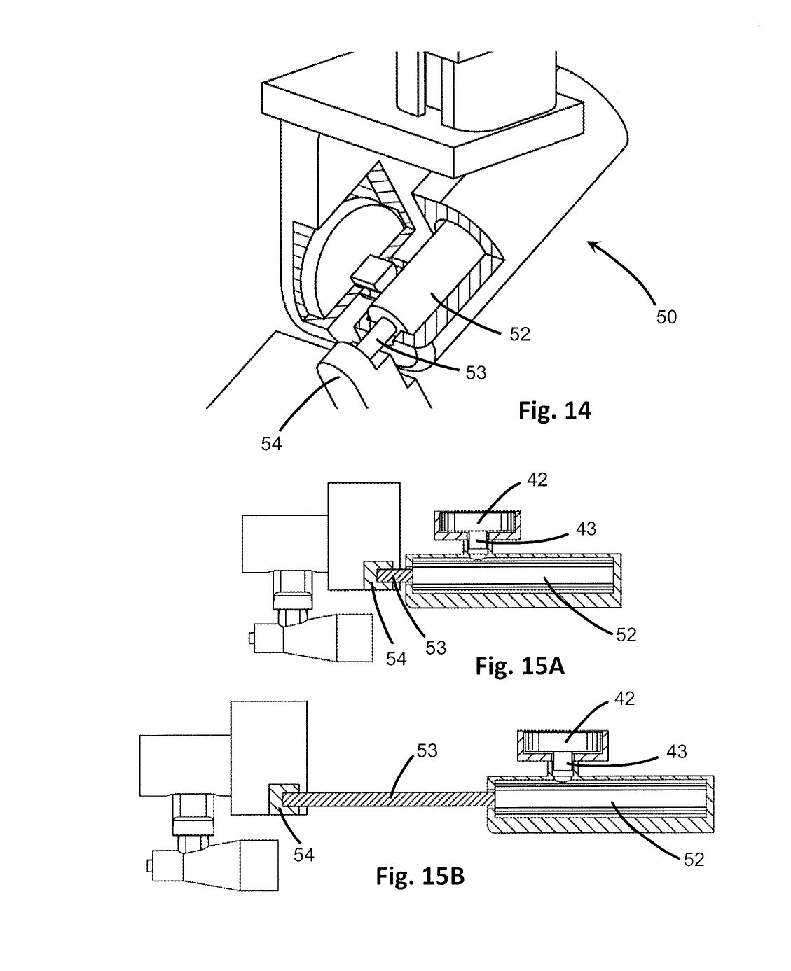

The positioner 120 further includes an extender 50, shown in FIGS. 7 and 13-15B. The extender 50 includes an extension actuator 52 in electrical communication with the processor 22 to receive extension adjustment data and instructions on activation and distance to move. When data are received, the extension actuator 52 activates to move the penetrating member 10 in a linear direction 51, as seen in FIG. 13, by a predetermined distance as calculated by the processor 22. In at least one embodiment, as shown in FIGS. 13-15B, the extension actuator 52 is a linear motor, although other forms of motors may be used for achieving movement of the penetrating member along the linear direction 51.

The extender 50 also includes an extension shaft 53 that extends out from the extension actuator 52 to an oppositely disposed extension mount 54 located on a separate component of the positioner 120. The extension shaft 53 may be secured to or integrally formed with the extension actuator 52, the extension mount 54, or both. The extension shaft 53 may retract into or be housed within the extension actuator 52 or share a common housing, and may be pushed out of the housing by the extension actuator. In some embodiments, as shown in FIG. 13, the extension shaft 53 may be a telescoping shaft. In other embodiments, as in FIGS. 15A and 15B, the extension shaft 53 may be a uniform bar or elongate member that is moved into and out of the extension actuator 52 upon activation. The distance the extension shaft 53 is pushed out of the extension actuator 52 is directed and calculated by the processor of the processor 22, based on the positioning information for the target point 29 input by the user on the display 24. The extension shaft 53 is made of a rigid material, such that as the extension shaft 53 is moved, the extension mount 54 in which it terminates is correspondingly moved. In this manner, the penetrating member 10 is moved the calculated distance in the linear direction 51 by the extension actuator 52, as shown in FIG. 13.

In some embodiments, the extension actuator 52 is used to move the penetrating member 10 a calculated distance to align it or otherwise position it for use, such as by moving it so the tip of the penetrating member 10 touches the skin or tissue 5 of the patient. In other embodiments, the extension actuator 52 is used to deploy the penetrating member 10 such that the tip of the penetrating member 10 moves from a ready position to the location of the target point 29. In at least one embodiment, the extension actuator 53 is used to both align and deploy the penetrating member 10 in a linear direction toward the target point 29. Both alignment and deployment of the penetrating member 10 may be automated. In at least one embodiment, deployment of the penetrating member 10 occurs as a result of activation of a button or particular area of the display 24, such as a soft button or virtual button on a touch screen, or button on a joystick or other part of the insertion device 100, which may be activated separately from the alignment and positioning of the penetrating member 10 in the other various dimensions by the user's placement of the detector 20 and the action of the vertical and angular actuators 32, 42.

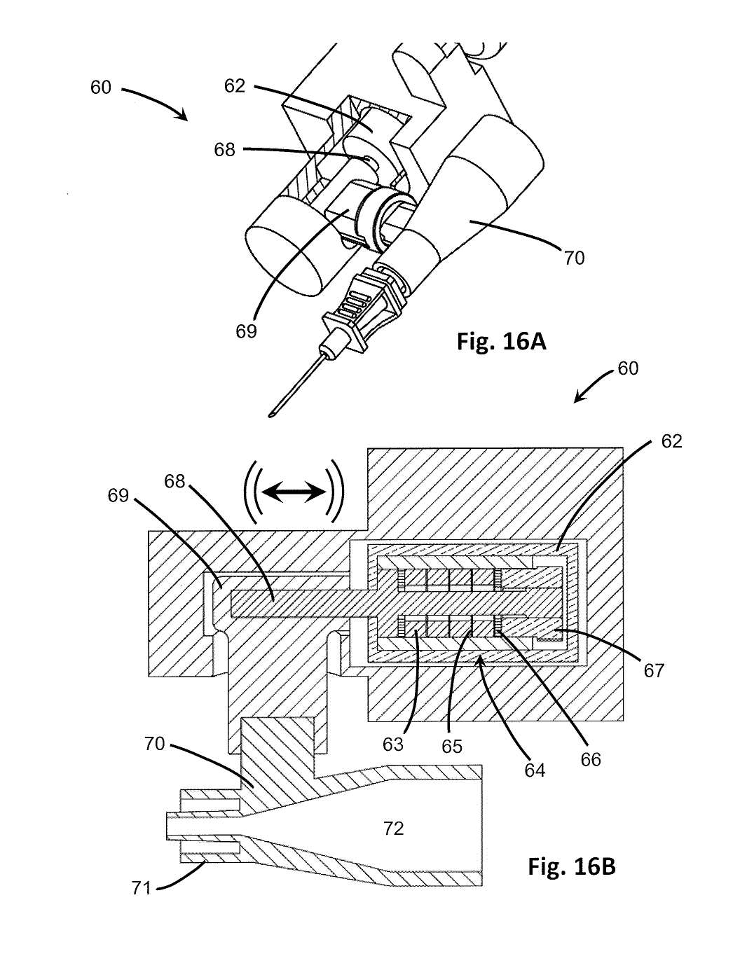

The insertion device 100 also includes a vibrator 60, for example as shown in FIGS. 7, 16A and 16B. The vibrator 60 includes a vibrational actuator 62 in electrical communication with the processor 22 and receives vibrational data from the processor 20 instructing when to activate and the operational parameters to use, which are determined by the processor 20 and may be based on a variety of factors, including but not limited to the type of vibrational actuator 62 used, and the type and condition of the tissue 5 being penetrated. When activated, the vibrational actuator 62 provides repetitive, reciprocating or oscillating motion to the penetrating member 10 back and forth along a longitudinal direction 61. The longitudinal direction 61 is coincident with the axis of the penetrating member 10. As used herein, the terms “reciprocating,” “oscillating,” and “vibrating” may be used interchangeably, and refer to a back and forth motion in a longitudinal direction 61 coincident with or parallel to the length of the penetrating member 10.

Upon receiving the activation signal from the processor 22, the vibrational actuator 62 turns on. Activation may occur automatically, or only at a certain point in the insertion process, such as once the penetrating member 10 is properly positioned and aligned but prior to being deployed for insertion. Activation of the vibrational actuator 62 may therefore occur only once the proper positioning of the penetrating member 10 is confirmed by a user in some embodiments, or may automatically begin once the target point 29 is aligned.

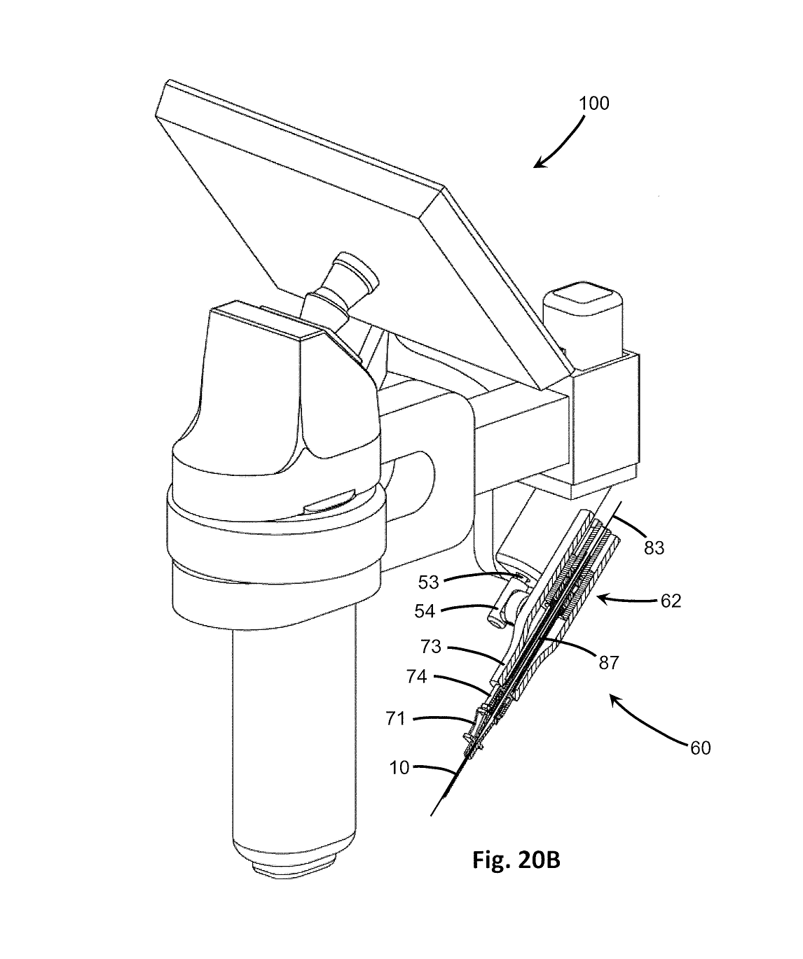

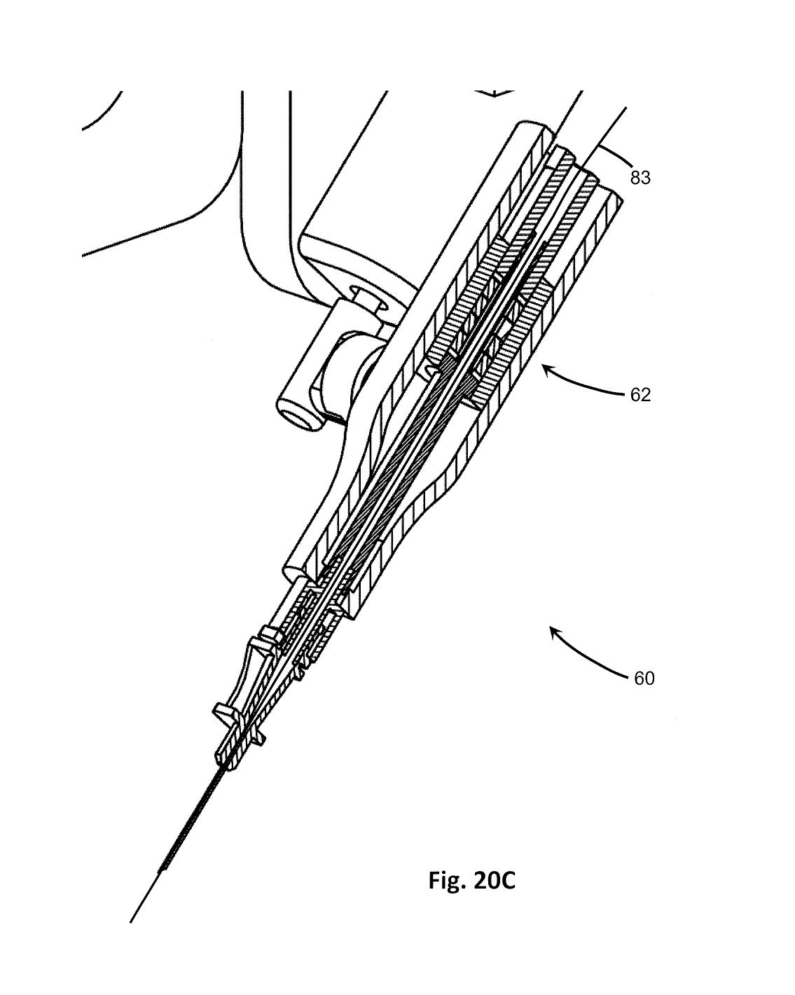

The vibrator 60 includes a drive shaft 68 that extends from the vibrational actuator 62 to a coupler or housing connected to the penetrating member 10. The drive shaft 68 transfers the mechanical vibrational motion generated by the vibrational actuator 62 to the penetrating member 10. The vibrator 60, and therefore the vibrational actuator 62, may be axially offset from the penetrating member 10 in some embodiments, as in FIGS. 16A and 16B, or may be inline or coaxial with the penetrating member 10, as in FIGS. 20A and 20B.

In at least one embodiment, as shown in FIGS. 16A and 16B, the vibrational actuator 62 is axially offset from the penetrating member 10. Here, the vibrating assembly 60 includes a drive shaft 68 that extends from the vibrational actuator 62 to a driving coupler 69. In some embodiments, the drive shaft 68 extends at least partially into the driving coupler 69. The driving coupler 69 coordinates with, such as by connecting to, an offset coupler 70. For instance, at least a portion of the driving coupler 69 may extend into the offset coupler 70, or vice versa. The offset coupler 70 includes a hub 71 at which a proximal end of the penetrating member 10 connects, such as by a screw, twist, threaded, or keyed connection, or other suitable connection. The driving coupler 69 and offset coupler 70 run perpendicular to the drive shaft 68 and the penetrating member 10. Therefore, the driving coupler 69 and offset coupler 70 collectively transfer the vibratory motion generated by the vibrational actuator 62 and propagated by the drive shaft 68 to the penetrating member 10 along a different, parallel axis.

In at least one other embodiment, as in FIGS. 20A and 20B, the vibrator 60′ and vibrational actuator 62′ of the insertion device 100′ is coaxial, or inline, with the penetrating member 10. In such embodiments, the drive shaft 68′ extends from the vibrational actuator 62′ to a portion of the housing 73. The housing 73 may include the vibrational actuator 62′ as well, and connects to a hub 71 a distal end where the penetrating member 10 connects. In some embodiments, the housing 73 may further include a neck 74 that extends between the housing 73 and the hub 71, such as if additional space is needed.

Regardless of whether the vibrator 60, 60′ is offset or inline with the penetrating member 10, vibration of the penetrating member 10 by the vibrational actuator 62 may be accomplished in a variety of ways, which may be selected based on the type of tissue being penetrated. The particular actuation mechanism useful to overcome the tissue deformation and insertion force depends on the resonance frequency and other electromechanical properties of the system to beneficially interact with the resonance and other mechanical properties of the tissue, vessels or other structures encountered by the advancing tip of the penetrating member 10.

For instance, in at least one embodiment, the vibrational actuator 62 is a piezoelectric motor. Transducer technologies that rely on conventional, single or stacked piezoelectric ceramic assemblies for actuation can be hindered by the maximum strain limit of the piezoelectric materials themselves. Because the maximum strain limit of conventional piezoelectric ceramics is about 0.1% for poly crystalline piezoelectric materials, such as ceramic lead zirconate titanate (PZT) and 0.5% for single crystal piezoelectric materials, it would require a large stack of cells to approach displacement or actuation of several millimeters or even many tens of microns. Using a large stack of cells to actuate components would also require that the medical tool size be increased beyond usable biometric design for handheld instruments.

Flextensional transducer assembly designs have been developed which provide amplification in piezoelectric material stack strain displacement. The flextensional designs comprise a piezoelectric material transducer driving cell disposed within a frame, platen, endcaps or housing. The geometry of the frame, platen, endcaps or housing provides amplification of the axial or longitudinal motions of the driver cell to obtain a larger displacement of the flextensional assembly in a particular direction. Essentially, the flextensional transducer assembly more efficiently converts strain in one direction into movement (or force) in a second direction.

Therefore, as shown in FIG. 16B, the vibrational actuator 62 is a flextensional transducer which includes a plurality of piezoelectric elements 63 stacked together with electrodes 65 placed between adjacent piezoelectric elements 63. The plurality of piezoelectric elements 63 and electrodes 65 stacked together form a piezoelectric stack 64. An insulator 66 caps the end of the stack 64 to shield the remainder of the device from the energy produced by the piezoelectric elements 63. A rear mass 67 located on the opposite side of the insulator 66 applies tension to the piezoelectric stack 64 and keeps the stack 64 compressed together for increased efficiency. At least the piezoelectric stack 64, and preferably the insulator 66 and rear mass 67 as well, are cylindrical and formed with a hollow bore running through the center. The drive shaft 68 extends through this hollow bore through the vibrational actuator 62. When the electrodes 65 are electrically stimulated, such as when the vibrational actuator 62 receives a signal from the processor 22 to activate, the electrical energy channeled through the electrodes 65 is converted into mechanical vibrational energy by the piezoelectric elements 63, which in turn is transferred to the drive shaft 68 to move the drive shaft 68 in a repetitive, oscillatory motion in the linear direction 61.

A variety of flextensional transducers are contemplated for use as the vibrational actuator 62, 62′. For example, in one embodiment, flextensional transducers are of the cymbal type, as described in U.S. Pat. No. 5,729,077 (Newnham), which is incorporated herein by reference. In another embodiment, flextensional transducers are of the amplified piezoelectric actuator (“APA”) type as described in U.S. Pat. No. 6,465,936 (Knowles), which is also incorporated herein by reference. In yet another embodiment, the transducer is a Langevin or bolted dumbbell-type transducer, similar to, but not limited to that which is disclosed in United States Patent Application Publication No. 2007/0063618 A1 (Bromfield), which is also incorporated herein by reference. FIG. 16B shows one particular example implementing a Langevin transducer as the vibrational actuator 62.

In one embodiment, the flextensional transducer assembly may utilize flextensional cymbal transducer technology or in another example, amplified piezoelectric actuator (APA) transducer technology. The flextensional transducer assembly provides for improved amplification and improved performance, which are above that of a conventional handheld device. For example, the amplification may be improved by up to about 50-fold. Additionally, the flextensional transducer assembly enables housing configurations to have a more simplified design and a smaller format. When electrically activated by an external electrical signal source, the vibrational actuator 62, 62′ converts the electrical signal into mechanical energy that results in vibratory motion of the penetrating member 10. The oscillations produced by the vibrational actuator 62, 62′ are in short increments (such as displacements of up to 1 millimeter) and at such a frequency (such as approximately 125-175 Hz) as to reduce the force necessary for puncturing and sliding through tissue, thereby improving insertion control with less tissue deformation and trauma, ultimately producing a higher vessel penetration/access success rate.

The vibratory motion produced by the vibrational actuator 62, 62′ creates waves, which may be sinusoidal waves, square waves, standing waves, saw-tooth waves, or other types of waves in various embodiments. In the case of a Langevin actuator, as in FIG. 16B, the vibratory motion produced by the piezoelectric elements 63 generates a standing wave through the whole assembly. Because at a given frequency, a standing wave is comprised of locations of zero-displacement (node, or zero node) and maximum displacement (anti-node) in a continuous manner, the displacement that results at any point along the vibrational actuator 62 depends on the location where the displacement is to be measured. Therefore, the horn of a Langevin transducer is typically designed with such a length so as to provide the distal end of the horn at an anti-node when the device is operated. In this way, the distal end of the horn experiences a large vibratory displacement in a longitudinal direction 61 with respect to the long axis of the vibrational actuator 62. Conversely, the zero node points are locations best suited for adding port openings or slots so as to make it possible to attach external devices.

In other embodiments, the vibrational actuator 62, 62′ may be a voice coil for the driving actuator rather than piezoelectric elements. Voice coil actuator (also referred to as a “voice coil motor”) creates low frequency reciprocating motion. The voice coil has a bandwidth of approximately 10-60 Hz and a displacement of up to 10 mm that is dependent upon applied AC voltage. In particular, when an alternating electric current is applied through a conducting coil, the result is a Lorentz Force in a direction defined by a function of the cross-product between the direction of current through the conductive coil and magnetic field vectors of the magnetic member. The force results in a reciprocating motion of the magnetic member relative to the coil support tube which is held in place by the body. With a magnetic member fixed to a driving tube, the driving tube communicates this motion to an extension member, such as a drive shaft 68, which in turn communicates motion to the penetrating member 10. A first attachment point fixes the distal end of the coil support tube to the body. A second attachment point fixes the proximal end of the coil support tube to the body. The magnetic member may be made of s Neodymium-Iron-Boron (NdFeB) composition. However other compositions such as, but not limited to Samarium-Cobalt (SmCo), Alnico (AlNiCoCuFe), Strontium Ferrite (SrFeO), or Barium Ferrite (BaFeO) could be used. Slightly weaker magnets could be more optimal in some embodiments, such as a case where the physical size of the system is relatively small and strong magnets would be too powerful.

The conducting coil may be made of different configurations including but not limited to several layers formed by a single wire, several layers formed of different wires either round or other geometric shapes. In a first embodiment of the conducting coil, a first layer of conductive wire is formed by wrapping the wire in a turn-like and spiral fashion and in a radial direction around the coil-support tube, with each complete revolution forming a turn next to the previous one and down a first longitudinal direction of the coil support tube. After a predetermined number of turns, an additional layer is formed over the first layer by overlapping a first turn of a second layer of the wire over the last turn of the first layer and, while continuing to wrap the wire in the same radial direction as the first layer, forming a second spiral of wiring with at least the same number of turns as the first layer, each turn formed next to the previous one and in a longitudinal direction opposite to that of the direction in which the first layer was formed. Additional layers may be added by overlapping a first turn of each additional layer of the wire over the last turn of a previous layer and, while continuing to wrap the wire in the same radial direction as the previous layer, forming an additional spiral of wiring with at least the same number of turns as the previous layer, each turn formed next to the previous one and in a longitudinal direction opposite to that of the direction in which the previous layer is formed. In an alternative voice coil embodiment, the locations of the magnetic member and conductive coil are switched. In other words, the conductive coil is wrapped around and attached to the driving tube and the magnetic member is located along an outside radius of the coil support tube. An electrical signal is applied at the conductive attachment sites and causes the formation of the Lorentz force to form in an alternating direction that moves the conductive coil and extension member reciprocally along the longitudinal axis of the device. The conductive coils are physically in contact with the driving tube in this embodiment.

In another embodiment, the vibrational actuator 62, 62′ employs a dual-coil mechanism in which the magnetic member of the voice-coil is replaced with a second conductive coil. In other words, the second conductive coil is wrapped around and attached to the driving tube and the first conductive coil is located along an outside radius of the coil support tube as before. In a first version, the inner coil conducts direct current DC and the outer coil conducts alternating current AC. In a second version, the inner coil conducts alternating current AC and the outer coil conducts direct current DC. In a third version, both the inner and outer coils conduct alternating current AC. In all of the voice coil actuator configurations described, springs may be used to limit and control certain dynamic aspects of the penetrating member 10.

In still another embodiment, the vibrational actuator 62, 62′ is a solenoid actuator. As with the other voice coil embodiments using coils, the basic principle of actuation with a solenoid actuator is caused by a time varying magnetic field created inside a solenoid coil which acts on a set of very strong permanent magnets. The magnets and the entire penetrating member assembly oscillate back and forth through the solenoid coil. Springs absorb and release energy at each cycle, amplifying the vibration seen at the penetrating member 10. The resonant properties of the vibrational actuator 62, 62′ can be optimized by magnet selection, number of coil turns in the solenoid, mass of the shaft, and the stiffness of the springs.

While piezoelectric and voice coil mechanisms have been discussed for the vibrational actuator 62, 62′, these are not the only approaches to actuating or oscillating the penetrating member 10. Other approaches, such as a rotating motor, could be used for the vibrational actuator 62, 62′. Generally, any type of motor comprising an actuator assembly, further comprising a mass coupled to a piezoelectric material, or a voice coil motor, or solenoid, or any other translational motion device, would also fall within the spirit and scope of the invention.

During use, feedback to track or confirm the vibrating tip of the penetrating member 10 has reached the desired target point 29 location may be obtained in several forms. First, the vibrating tip of the penetrating member 10 may be visualized on the display 24 as its echo is picked up by the detector 20 during ongoing imaging through the insertion process. This can be performed while viewing the image in long-axis view or short-axis view (as in FIG. 5C), or a user may toggle between long and short-axis views as desired to follow the progress of the tip of the penetrating member 10. Second, the appearance of fluid, such as blood, in the penetrating member, also referred to as “flashback,” may be detected through mechanisms such as visual identification, change in resistance to a sub-circuit, or change in resonance frequency or phase of the vibrating needle tip, to name but a few. Other methods of confirming the tip of the penetrating member 10 has reached the preselected target point 29 may also be used.

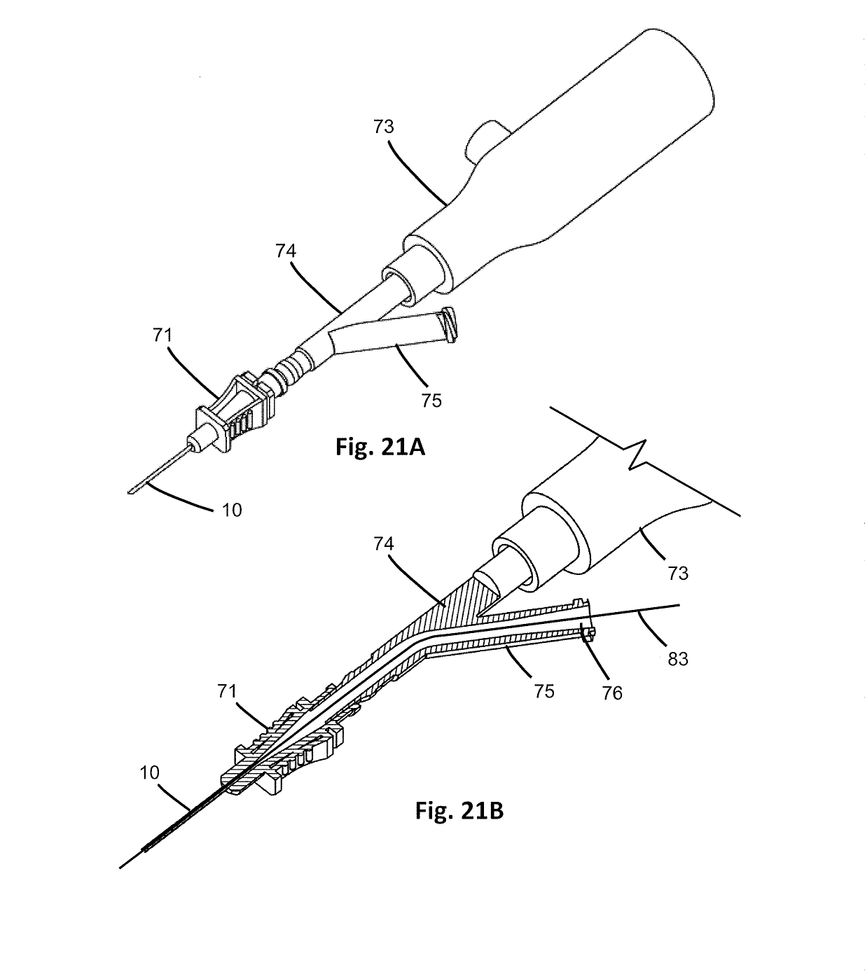

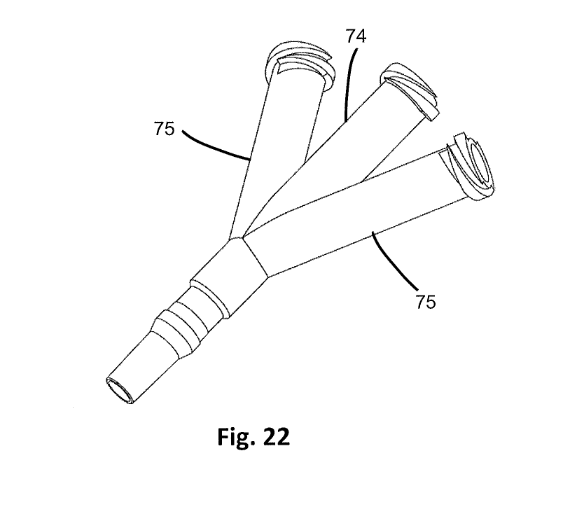

After the tip of the penetrating member 10 is successfully inserted in the target vessel and positioned at the desired target point 29, the remainder of the procedure for successful central venous catheterization, discussed above according to the Seldinger technique, could be accomplished. For instance, in one embodiment, a guidewire 83 may be fed through the penetrating member 10 for insertion into the target vessel. The penetrating member 10 may therefore be dimensioned to accommodate a guidewire 83, having an inner diameter at least as large as the diameter of a guidewire 83 which is to be inserted therein. For instance, in some embodiments the penetrating member 10 may be between 14 and 18 gauge, while the outer diameter of the guidewire 75 may range of 0.9 to 0.6 millimeters (0.035-0.024 inches). Of course, other sizes and gauges are also contemplated herein. The guidewire 83 may be extended beyond the tip of the penetrating member 10 by 1-3 cm, although shorter and longer distances for guidewire insertion are also contemplated. For instance, the guidewire 83 may be fed through an interior 72 volume or space of the offset coupler 70 that has an opening in alignment with the hub 71, and therefore, penetrating member 10, as seen in FIG. 16B. In other embodiments, as in FIGS. 21A-22, the guidewire 83 may be fed through a lumen 76 in a side port(s) 75 at the housing 73 of the vibrator 60′, such as the neck 74 before the hub 71. The housing 73, neck 74, sideport(s) 75 and hub 71 may all be integrally formed together, or may all be separate components that are selectively attachable to each other, such as with a Luer connection or other suitable selectively removable connection mechanism, or any combination thereof. For instance, in some embodiments, the sideport(s) 75 is integrally formed with the neck 74, which is attachable to the housing 73 on one end and the hub 71 on the opposite end, as shown in FIG. 21B. Accordingly, the neck 74 and sideport 75 may be a Wye adaptor. In other embodiments, the sideport(s) 75 may be separate from and attach to the housing 73 or neck 74. In still other embodiments, the neck 74, sideport(s) 75 and hub 71 may be integrally formed, and connect to the housing 73.

Once the guidewire 83 is inserted through the penetrating member 10 and placed as desired in the target vessel, the penetrating member 10 may then be retracted from the vessel, such as by the extension actuator 52 moving in the reverse direction along the linear direction 51, leaving the guidewire 83 in place. A dilator may also be inserted and retracted as needed to expand the space. A catheter may then be inserted over the guidewire, and the guidewire retracted from the vessel, leaving the catheter in place.

The vertical actuator 32, angular actuator 42, extension actuator 52 and vibrational actuator 62 are integrated in the insertion device 100. Accordingly, in at least one embodiment, the penetrating member 10 may be selectively removable from the insertion device 100, such as by attachment and detachment at the hub 71, so that a sterile penetrating member 10 may be used with each new patient or use. Accordingly, the penetrating member 10 may be disposable and the rest of the insertion device 100, including the detector 20, processor 22, and various actuators 32, 42, 52, 62, all remain intact and are reusable.

In at least one embodiment, at least a portion of but preferable the entire insertion device 100 up to and including the hub 71 is reusable and may be included in a sterility bag to maintain sterile conditions. In some embodiments, the sterility bag may be wiped down, such as with alcohol or bleach, between patients or uses, such that full sterility measures do not need to be taken on the reusable insertion device 100 between uses every time. In other embodiments, the hub 71 may be removable from the offset coupler 70 or housing 73 for sterilization between uses or disposal. In still other embodiments, the offset coupler 70 or housing 70 may be removable from the remainder of the device 100, 100″ for sterilization between uses or disposal. Throughout the various embodiments, it is contemplated that the reusable portions of the insertion device 100, 100″ may be encased in a sterility bag or like structure to maintain sterile conditions between use.

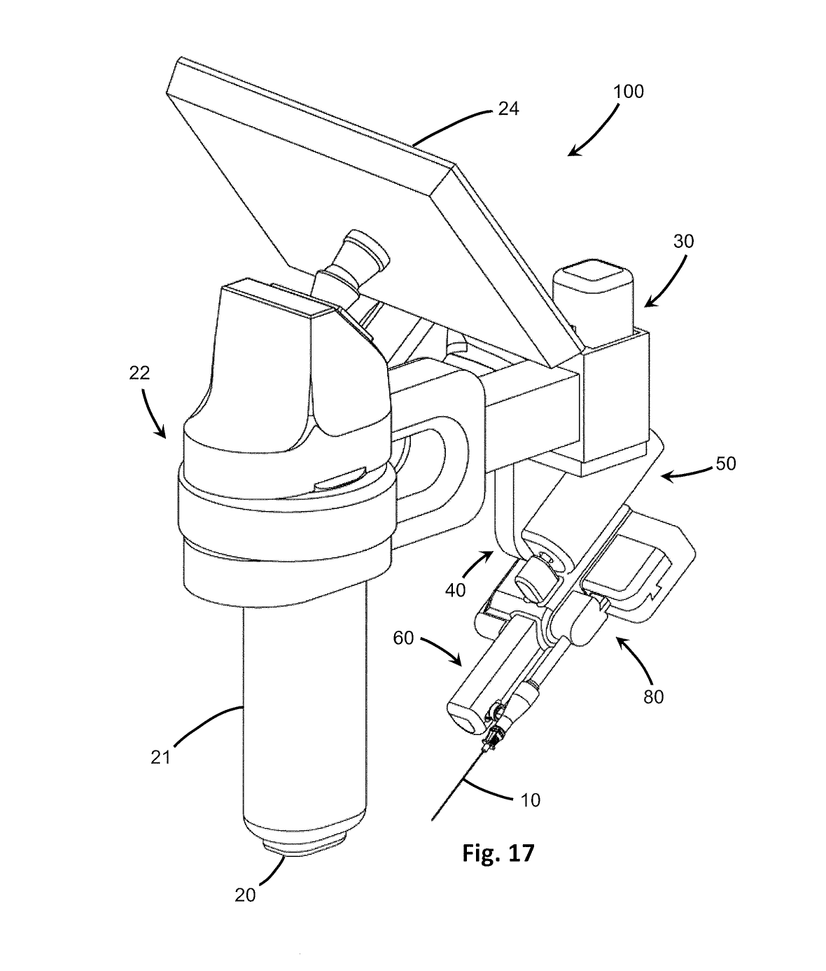

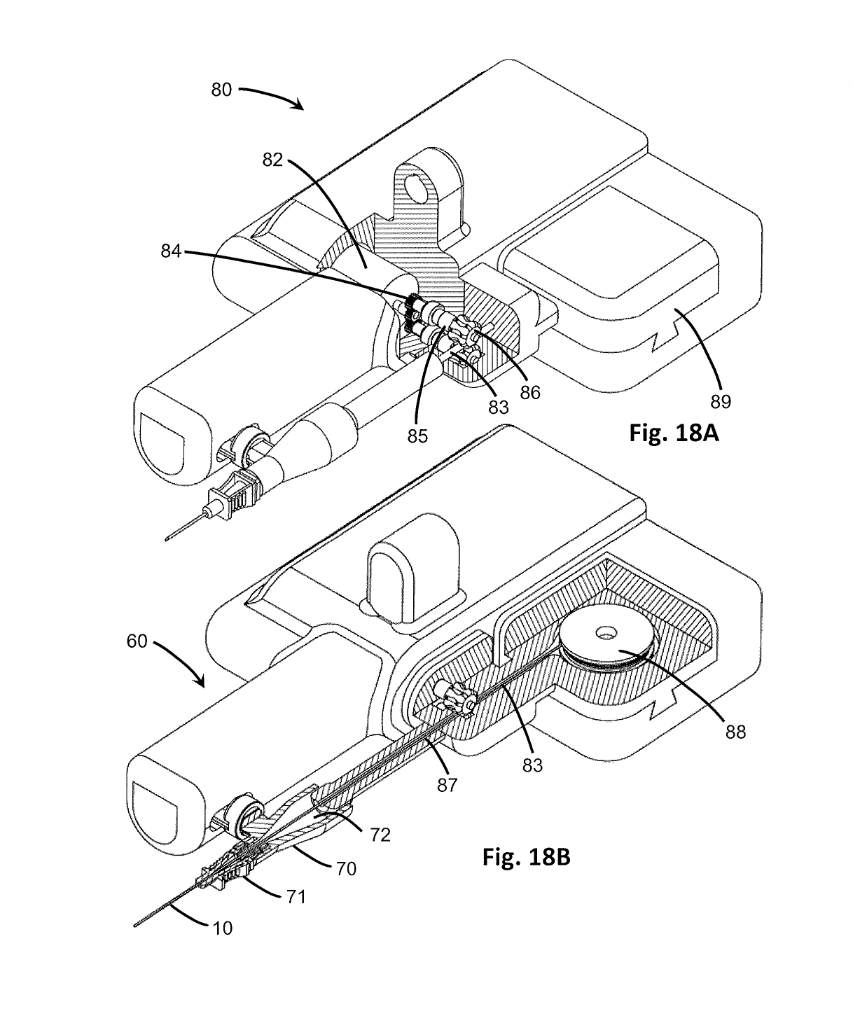

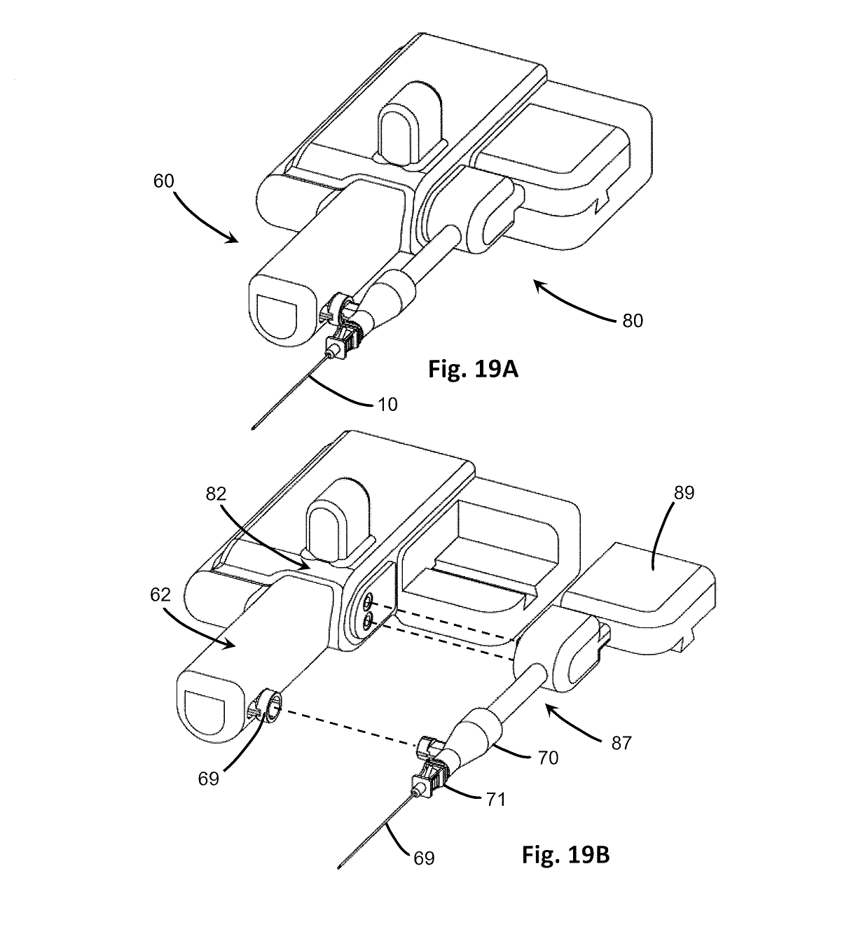

In at least one embodiment, as shown in FIGS. 17-19B, the insertion device 100′ may include a guidewire adjustment 80 for inserting a guidewire 83 as directed by the processor 22. A guidewire actuator 82 is in electrical communication with the processor 22 and receives operative data from the processor 22 directing activation and operational parameters based on the type of actuator, location of guidewire, etc. For instance, in at least one embodiment shown in FIGS. 18A and 18B, the guidewire actuator 82 is a rotational motor, which may have at least one, but in some instances, two elongate members 85 that extend from the guidewire actuator 82. A gear(s) 84 of the guidewire actuator 82 turns at least one of the elongate member(s) 85. In some embodiments, only one elongate member 85 is active, being primarily engaged by the gear 84 for turning or rotating. Another elongate member 85 may also be present, such as paired with the first active elongate member, but may be passive such that it is not rotated by the guidewire actuator 82. Accordingly, a passive elongate member 85 may only rotate by action in response to movement of a paired active elongate member 85, such as by interdigitation of teeth on coordinating gears 84 between the elongate member 85.

Opposite from the guidewire actuator 82, the elongate member(s) 85 include a frictional member 86. In at least one embodiment, each elongate member 85 includes a frictional member 86, which may be at the terminal end of the elongate member 85. In other embodiments, only the primary elongate member 85 includes a frictional member 86, although preferably both active and passive elongate members 85 include their own respective frictional members 86. In embodiments where there are multiple active elongate members 85, each one includes a frictional member 86. The frictional member(s) 86 grip the guidewire 83 and using frictional engagement, move the guidewire 83 as they rotate. Some embodiments, as shown in FIGS. 18A and 18B, the guidewire 83 may be attached and enclosed in a guidewire housing 89, keeping the guidewire 83 sterile when not in use. In some embodiments, the guidewire 83 is retained as a spool 88 within the housing 89 for compact storage and easy unwinding when needed. In other embodiments, the guidewire 83 may extend out from the insertion device 100′ and may be fed through the device 100′ as needed. Regardless of whether coiled in a spool or not, as the guidewire actuator 82 turns the elongate member(s) 85, the frictional member(s) 86 engage the guidewire 83 and turn to move the guidewire 83, either advancing or retracting the guidewire, depending on the direction of rotation.

The guidewire 83 is moved through a guidewire channel 87 in the guidewire housing 89. The guidewire channel 87 is aligned with and in fluid communication with the interior 72 of the offset coupler 70, such that the guidewire 83 is advanced through the channel 87, through the interior 72 of the offset coupler 70, hub 71, and penetrating member 10. The guidewire 83 may be advanced beyond the tip of the penetrating member 10, as described previously. The guidewire 83 may be retracted through the same route and mechanism of the insertion device 100′, but rotating the elongate member(s) 85 and frictional member(s) 86 in the opposite direction.

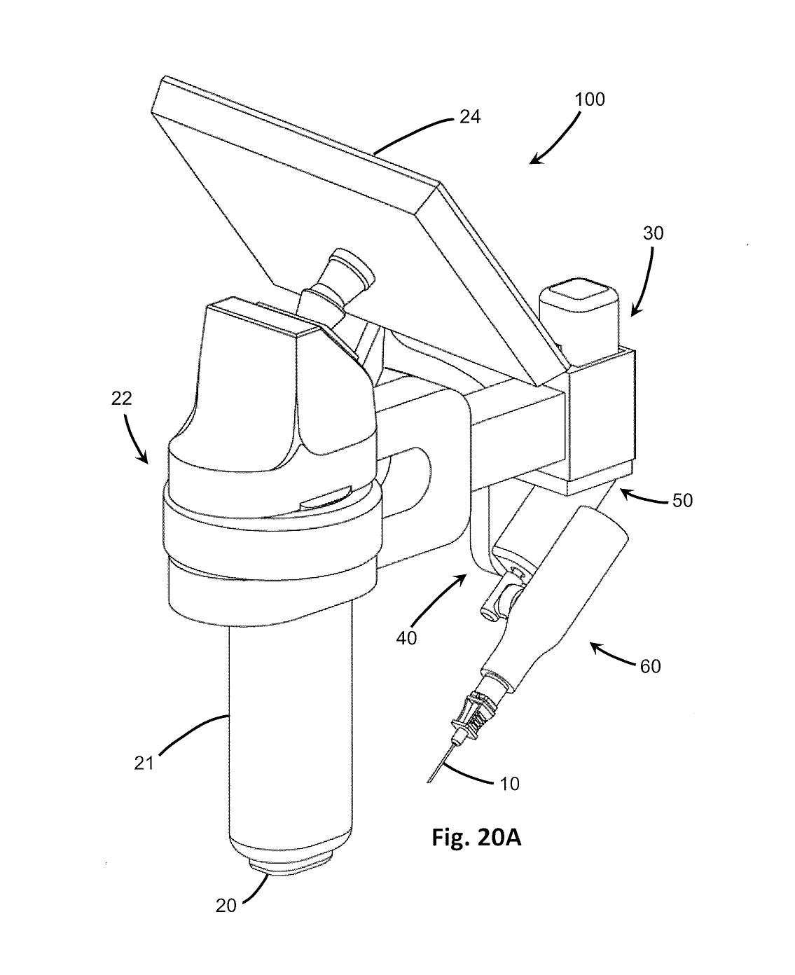

The guidewire 83 must also be sterile for use. Accordingly, in some embodiments, such as shown in FIGS. 19A and 19B, anything that the guidewire 83 touches may be selectively detachable and disposable, such as for one-time use. For instance, the guidewire housing 89 containing the spool 88, together with the guidewire channel 87, offset coupler 70, hub 71 and penetrating member 10 may all be separable from the remainder of the insertion device 100′, such that the detector 20, processor 22, and actuators 32, 42, 52, 62, and 82 all remain sterile and reusable. This is one benefit to having an offset alignment of the penetrating member 10 from the vibrational actuator 62. In other embodiments, just the guidewire 83 and penetrating member 10 may be removable and disposable, and the guidewire channel 87, offset coupler 70 and hub 71 may be sterilized between uses.

In still other embodiments, such as depicted in FIG. 20C, the guidewire 83 passes through the vibrational actuator 62. In such embodiments, the vibrational actuator 62 and the drive shaft 68 may have aligned lumens extending therethrough which act as a guidewire channel 87. The guidewire 83 may be advanced and retracted through these lumens.

Since many modifications, variations and changes in detail can be made to the described preferred embodiments, it is intended that all matters in the foregoing description and shown in the accompanying drawings be interpreted as illustrative and not in a limiting sense. Thus, the scope of the invention should be determined by the appended claims and their legal equivalents. Now that the invention has been described,

What is claimed is:

1. A system for penetrating tissue having a surface, comprising:
a detector that obtains data representative of the location of said tissue;
a penetrating member;
a vibrational actuator for inducing vibration in said penetrating member;
a positioner for adjusting the position of said penetrating member, including:
a vertical actuator for performing a vertical adjustment of said penetrating member,
an angular actuator for performing an angular adjustment of said penetrating member, and
an extension actuator for performing an extension adjustment of said penetrating member;
a processor in electronic communication with said detector, said vibrational actuator and said positioner, said processor:
(i) receiving said data representative of the location of said tissue from said detector;
(ii) calculating the distance between a preselected target point within said tissue and said tissue surface;
(iii) calculating vertical, angular and extension adjustment data for said vertical, angular and extension actuators, respectively, based upon said calculated distance between a preselected target point within said tissue and said tissue surface;
(iv) determining vibrational data based upon operative parameters of said vibrational actuator;
(v) transmitting said angular and extension adjustment data to said positioner; and
(vi) transmitting said vibrational data to said vibrational actuator;
causing said penetrating member to be inserted to said preselected target point within said tissue.

2. The system as recited in claim 1, wherein said data representative of said location of said tissue includes image data.

3. The system as recited in claim 2, further comprising a display presenting said image data of said location of said tissue.

4. The system as recited in claim 3, wherein said display further presents a visual representation of said preselected target point within said tissue.

5. The system as recited in claim 4, wherein said display is interactive and said visual representation of said preselected target point within said tissue can be moved around on said display.

6. The system as recited in claim 1, wherein said tissue includes a cavity below said tissue surface, said preselected target point is within said cavity within said tissue and below said tissue surface, and said detector obtains data representative of the location said preselected target point within said cavity, and said processor:
(i) receiving said data representative of the location of said preselected target point within said cavity from said detector;
(ii) calculating the distance between said preselected target point within said cavity and said tissue surface;
(iii) calculating vertical, angular and extension adjustment data for said vertical, angular and extension actuators, respectively, based upon said calculated distance between a preselected target point within said cavity and said tissue surface;
(iv) determining vibrational data based upon operative parameters of said vibrational actuator;
(v) transmitting said angular and extension adjustment data to said positioner; and
(vi) transmitting said vibrational data to said vibrational actuator;
causing said penetrating member to be inserted to said preselected target point within said cavity.

7. The system as recited in claim 6, wherein said detector utilizes sound waves to obtain said data representative of said location of said cavity within said tissue.

8. The system as recited in claim 7, wherein said detector utilizes ultrasound to obtain said data representative of said location of said cavity within said tissue.

9. A device for inserting a penetrating member into tissue, comprising:
a detector;
a vibrational actuator in mechanical communication with said penetrating member; and
a positioner including:
a vertical actuator for adjusting the distance between said penetrating member and said tissue in a vertical direction,
an angular actuator for adjusting the angle of inclination of said penetrating member to said tissue, and
an extension actuator for adjusting the distance between said penetrating member and said tissue in a linear direction;
wherein said positioner is mechanically interconnected with said penetrating member.

10. The device as recited in claim 9, wherein said vibrational actuator is a piezoelectric motor.

11. The device as recited in claim 10, wherein said vibrational actuator includes at least one of a flextensional transducer, a cymbal transducer, a Langevin transducer, an amplified piezoelectric actuator transducer.

12. The device as recited in claim 9, wherein said vibrational actuator is a voice coil motor.

13. The device as recited in claim 12, wherein said vibrational actuator includes at least one of a single conductive coil, dual conductive coils, and a solenoid actuator.

14. The device as recited in claim 9, further comprising a handle for gripping by a user, such that said device can be held with a single hand.

15. A method for inserting a penetrating member into tissue having a surface, comprising:
obtaining data representative of the location of a preselected target point within said tissue;
calculating the distance between said preselected target point within said tissue and said tissue surface;
calculating vertical, angular, and extension adjustment data for said penetrating member based on said calculated distance between said preselected target point within said tissue and said tissue surface;
adjusting the position of said penetrating member according to said calculated vertical, angular, and extension adjustment data;
determining vibrational data for said penetrating member;
inducing vibration in said penetrating member according to said determined vibrational data; and
inserting said penetrating member into said tissue to said preselected target point according to said calculated extension adjustment data.

16. The method as recited in claim 15, further comprising selecting a new target point prior to inserting said penetrating member into said tissue; calculating an updated distance between said new target point within said tissue and said tissue surface; calculating updated vertical, angular, and extension adjustment data for said penetrating member based on said calculated distance between said new target point within said tissue; and adjusting the position of said penetrating member according to said updated calculated vertical, angular, and extension adjustment data.

17. The method as recited in claim 16, wherein said calculating updated vertical, angular, and extension adjustment data for said penetrating member based on said calculated distance between said updated target point within said tissue and adjusting the position of said penetrating member according to said updated calculated vertical, angular, and extension adjustment data occur in real time as said new target point is selected.

18. The method as recited in claim 17, wherein inserting said penetrating member into said tissue to said new target point according to said updated extension adjustment data.
